SEQUENCE LISTING (14 sequences)

SEQ ID NO: 1 - protein, 119 aa, Homo Sapiens
QVQLVQSGGGVVQPGRSLRLSCKASGYTFTSYVMHWVRQAPGKGLEWIGYIVPYNDGTKYNEKFKGRFTISSDKSKSTAFLQMDSLRPEDTAVYYCARGSRYDWYLDYWGQGTPVTVSS

SEQ ID NO: 2 - protein, 113 aa, Homo Sapiens
NIMMTQSPSSLSASVGDRVTITCKSSQSVLYSSNQKNYLAWYQQTPGKAPKLLIYWASTRESGVPSRFSGSGSGTDYTFTISSLQPEDIATYYCHQYFSSYTFGQGTKLQITR

SEQ ID NO: 3 - protein, 119 aa, Mus Musculus
EVQLQQSGPELVKPGASVKMSCKASGYTFTSYVMHWVKQKPGQGLDWIGYIVPYNDGTKYNEKFKGKATLTSDKSSSTAYMELSRLTSEDSAVYYCVYGSRYDWYLDVWGAGTTVTVSS

SEQ ID NO: 4 - protein, 113 aa, Mus Musculus
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYFSSYTFGGGTKLEIKR

SEQ ID NO: 5 - DNA, 357 bp, Homo Sapiens
caggtgcagctggtgcagtctggcggtggagtggtccagcccggccgcagcctgaggctgtcctgcaaggcatctggctacaccttcaccagctacgtgatgacatgggtgcgccaagcccccggaaagggcctcgaatggattggctacattgtgccttataatgacggtactaagtacaatgaaaagttcaagggcagatttacaatatcaagtgacaagagcaagtcaaccgcattcctccaaatggacagcttgcgtccagaggacaccgccgtatactattgtgtgcgcggcagccgttacgactggtacttggactactggggccaaggcactccagtcaccgtctcctct

SEQ ID NO: 6 - DNA, 339 bp, Homo Sapiens
aacatcatgatgactcagagcccatccagcttgagcgcatcagtaggcgaccgcgtaacgatcacttgcaaatcctctcagtcagtattgtactccagcaaccagaagaactacctggccggatatcagcagactcccggcaaagccccaaagttgctgatttattgggcctccacgcgcgagtctggcgtgccatcacgctttagcggcagcgggtccggtacagattacacgtttaccattagcagtctgcagcctgaggacatagccacctactactgtcaccagtactttagttcctacacttttggccagggaactaaactgcagattactcga

SEQ ID NO: 7 - DNA, 357 bp, Mus Musculus
gaggtccagctgcagcagtctggacctgagctggtaaagcctggggcttcagtgaagatgtcctgcaaggcttctggatacacattcactagctatgttatgcactgggtgaagcagaagcctgggcagggccttgactggattggatatattgttccttacaatgatggcactaagtacaatgagaagttcaaaggcaaggccacactgacttcagacaaatcctccagcacagcctacatggagctcagcagactgacctctgaggactctgcggtctattattgtgtctacggtagtaggtacgactggtatttagatgtctggggcgcagggaccacggtcaccgtctcctca

SEQ ID NO: 8 - DNA, 339 bp, Mus Musculus
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcagcagtgtacaagctgaagacctggcagtttattactgtcatcaatatttctcctcatacacgttcggaggggggaccaagctggaaataaagcgg

SEQ ID NO: 9 - protein, 119 aa, Homo Sapiens
QVQLVESGGGVVQPGCSLRLSCSSSGYTFTSYTMHWVRQAPGKGLEWIGYINPYNDGGKYNEKFKWRFSISSDKSKNTLFLQSDSLTPEDTGVYYCARGSRYDWYGDYWGQGTPVTVSS

SEQ ID NO: 10 - protein, 113 aa, Homo Sapiens
DIQMTQSPSSLSGSVGDRVTITCDSSQSVLYSSKDDNYLAWYQQGPGKAPSLLIYYASDRESDVPSRFSGSGSGDDYTLTISSLQPEDAATYYCHQWFSSYTFDQGTKLNITR

SEQ ID NO: 11 - DNA, 357 bp, Homo Sapiens
caggtgcagctggtggagtctggcggtggagtggtccagcccggctgcagcctgaggctgtcctgcagtagctctggctacaccttcaccagctacaccatgacatgggtgcgccaagcccccggaaagggcctcgaatggattggctacattaatccttataatgacggtgggaagtacaatgaaaagttcaagtggagattttcaatatcaagtgacaagagcaagaacaccctgttcctccaaagcgacagcttgaccccagaggacaccggcgtatactattgtgtgcgcggcagccgttacgactggtacggggactactggggccaaggcactccagtcaccgtctcctct

SEQ ID NO: 12 - DNA, 339 bp, Homo Sapiens
gacatccagatgactcagagcccatccagcttgagcggctcagtaggcgaccgcgtaacgatcacttgcgactcctctcagtcagtattgtactccagcaaagacgacaactacctggccggatatcagcaggggcccggcaaagccccaagcttgctgatttattatgcctccgaccgcgagtctgacgtgccatcacgctttagcggcagcgggtccggtgatgattacacgctgaccattagcagtctgcagcctgaggacgccgccacctactactgtcaccagtggtttagttcctacacttttgaccagggaactaaactgaacattactcga

SEQ ID NO: 13 - protein, 22 aa, Homo Sapiens
MDFQVQIFSFLLISASVIISRG

SEQ ID NO: 14 - DNA, 67 bp, Homo Sapiens
atggattttcaggtgcagattttcagcttcctgctaatcagtgcctcagtcataatatccagaggag

(19) United States
(12) Patent Application Publication
(10) Pub. No.: US 2005/0031617 A1
(43) Pub. Date: Feb. 10, 2005

(54) Antibodies specific for cancer associated antigen SM5-1 and uses thereof

(76) Inventors: Jing Ma, San Diego, CA (US); Yajun Guo, Shanghai (CN)

Correspondence Address: Barry Wilson;Foley & Lardner, LLP, Suite 200, 11250 El. Camino Real, San Diego, CA, 92130, US

(21) Appl. No.: 10/722,849

(22) Filed: Nov. 26, 2003

(30) Foreign Application Priority Data
Jun. 6, 2003 (CN) 03129123.6
Nov. 25, 2003 (CN) 200310119926.4

(51) Int. Cl. A61K 39/395; C07K 16/40

(52) U.S. Cl. 424/146.1; 530/388.26

(57) ABSTRACT

The invention concerns antibodies which is specific for SM5-1 antigen expressed in melanoma, breast cancer and hepatocellular carcinoma, and polynucleotides encoding the antibodies. The invention further concerns use of such antibodies and/or polynucleotides in diagnosing and treating malignancies.

### CROSS-REFERENCE TO RELATED APPLICATIONS

This application claims the benefit of Chinese application serial nos. 03129123.6, filed Jun. 6, 2003, and ______, filed Nov. 25, 2003 (title: Antibodies specific for cancer associated antigen SM5-1 and uses thereof) which are incorporated in their entirety by reference.

### BACKGROUND OF THE INVENTION

1. Field of the Invention

The present invention relates generally to the field of cancer biology and immunotherapy. More specifically, it relates to a tumor antigen specifically expressed in melanoma, breast cancer and hepatocellular carcinoma, antibodies directed against the tumor antigen, and methods of diagnosing and/or treating cancers associated with the antigen.

2. Description of the Related Art

Malignancy is one of the key diseases threatening the life of human kind. Melanoma, breast cancer and hepatocellular carcinoma are some of the most common malignancies.

Melanoma is a type of cancer with high degree of malignancy. About 20% of the melanoma cases occur in neck and head, with the majority on skin and mucus membranes of cavities and sinuses. Generally human melanomas originate from pigmentary naevi, which become destructive metastatic melanoma cells when stimulated by irradiation. Melanomas are quite liable to spread through the body and hard to control. Chemotherapy and radiotherapy are not effective to melanomas.

The most commonly used antibodies for melanoma immunohistochemical diagnosis are HMB-45, anti-S—I 00, and NKI/C3. (Smoller B R, Pathology: state of the art reviews, 2: 371-383, 1994). These antibodies have their disadvantages. The sensitivity of HMB-45 is not high enough (sensitivity between 67%-93%), and may leave some melanoma cases undiagnosed (Wick M R et al., J Cutan Pathol 15: 201-207, 1988; Ordonez N G et al. Am J. Clin. Pathol. 90: 385-390, 1988). Although anti-S-100 antibody is more sensitive compared to HMB-45 (Nakajima T et al., Cancer 50: 912-918, 1982; Kindblom L G et al., Acta. Pathol. Microbial. Immunol. Scand 92: 219-230, 1984), it is not very specific. NKI/C3 binds to a lot of benign as well as malignant tumors, and this non-specificity greatly limits the application of NKI/C3 in melanoma diagnosis.

Breast cancer is one of the most frequent malignancies for women. About 1.2 million women are affected by the disease each year, and 0.5 million are dying from the disease each year in the world. Developed countries in North America, Western and Northern Europe have the highest occurrence of the disease, and countries in Africa have the lowest occurrence. The new cases of breast cancer are increasing significantly all across the world and are growing at a rate of 5-20% both in high-occurring and low-occurring areas.

Hepatocellular carcinoma is one of the most frequent malignancies in China and its occurrence is related to hepatitis. Liver cancer antigens that are most studied include PHC, F062, 25T and Hab18 et al (Lian-Jun Yang, Wen-Liang Wang., World J. Gastroenterol. 8(5): 808-814, 2002). AFP is found not only expressed in the blood of liver cancer patients, but also expressed in ovarian and testis cancer cells. Hepatoma-specific monoclonal antibody Hab18 has also been studied, but it fails to be an effective target for hepatoma therapy.

Although many monoclonal antibodies have been developed for immunotherapy of malignancies, the specificity and neutralizing capacity of these monoclonal antibodies are not ideal. There is a need to develop monoclonal antibodies (including humanized antibodies) with high specificity and high neutralizing capacity for malignant tumors.

### SUMMARY OF THE INVENTION

The invention disclosed herein concerns antibodies (such as monoclonal antibodies) and polypeptides that specifically bind to an antigen SM5-1 which is expressed in melanoma, breast cancer, and hepatocellular carcinoma. The molecular weight of this antigen is 230 kD and 180 kD. This antigen can be isolated from a melanoma, breast cancer and/or hepatocellular carcinoma cell using antibodies of the invention.

In one aspect, the invention provides a human SM5-1 specific monoclonal antibody (huSM5-1). The variable region of heavy chain and light chain of the human SM5-1 specific monoclonal antibody (huSM5-1) are shown in Table 1. The antibody huSM5-1 is produced by a host cell having an accessing number ______ or progeny thereof.

In some embodiments, the invention is an antibody or a polypeptide comprising a fragment or a region of the antibody huSM5-1. In one embodiment, the fragment is a heavy chain of the antibody huSM5-1 as shown in Table 1. In another embodiment, the fragment is a light chain of the antibody huSM5-1 as shown in Table 1. In yet another embodiment, the fragment contains one or more variable regions from a heavy chain and/or a light chain of the antibody huSM5-1 as set forth in SEQ ID NO:9 and SEQ ID NO:10. In yet another embodiment, the fragment contains one or more complementarity determining regions (CDRs) from a heavy chain and/or a light chain of the antibody huSM5-1 as shown in Table 1.

In another aspect, the invention provides antibodies that competitively inhibits the immunospecific binding of antibody huSM5-1 to a SM5-1 target antigen. In some embodiments, the variable region of heavy chain of the antibody comprises the amino acid sequences 31-35, 50-66 and 99-108 set forth in SEQ ID NO:9 and the variable region of light chain of the antibody comprises the amino acid sequences 24-40, 56-62 and 95-102 set forth in SEQ ID NO:10. In other embodiments, the variable region of heavy chain of the antibody comprises the amino acid sequence set forth in SEQ ID NO:9. In yet other embodiments, the variable region of light chain of the antibody comprises the amino acid sequence set forth in SEQ ID NO:10. In yet other embodiments, the variable region of heavy chain of the antibody comprises the amino acid sequence set forth in SEQ ID NO:9 and the variable region of light chain of the antibody comprises the amino acid sequence set forth in SEQ ID NO: 10. In some embodiments, the antibody is a human antibody.

In another aspect, the invention provides a mouse SM5-1 specific antibody (mSM5-1) (the variable region of the heavy chain and the light chain are shown in Table 2). The variable region of heavy chain of antibody mSM5-1 comprises the amino acid sequence set forth in SEQ ID NO:3 and the variable region of light chain of the antibody mSM5-1 comprises the amino acid sequence set forth in SEQ ID NO:4.

In some embodiments, the invention is an antibody or a polypeptide comprising a fragment or a region of the antibody mSM5-1. In some embodiment, the fragment contains one or more variable regions from a heavy chain and/or a light chain of the antibody mSM5-1 as set forth in SEQ ID NO:3 and SEQ ID NO:4. In yet another embodiment, the fragment contains one or more complementarity determining regions (CDRs) from a heavy chain and/or a light chain of the antibody mSM5-1 as set forth in SEQ ID NO:3 and SEQ ID NO:4.

In another aspect, the invention provides an antibody that competitively inhibits the immunospecific binding of the antibody mSM5-1 to a SM5-1 target antigen. In some embodiments, the variable region of heavy chain of the antibody comprises the amino acid sequences 31-35, 50-66 and 99-108 set forth in SEQ ID NO:3 and the variable region of light chain of the antibody comprises the amino acid sequences 24-40, 56-62 and 95-102 set forth in SEQ ID NO:4. In some embodiments, the antibody is a humanized antibody.

In yet another aspect, the antibody immunospecific for a SM5-1 target antigen is a chimeric antibody, wherein the variable region of heavy chain of the antibody comprises the amino acid sequence set forth in SEQ ID NO:3 and the variable region of light chain of the antibody comprises the amino acid sequence set forth in SEQ ID NO:4.

In yet another aspect, the antibody of the invention is a humanized antibody, wherein the variable region of heavy chain of the humanized antibody comprises the amino acid sequence set forth in SEQ ID NO: 1 and/or the variable region of light chain of the humanized antibody comprises the amino acid sequence set forth in SEQ ID NO:2. In some embodiments, the humanized antibody is produced by a host cell with an accession number ______ or progeny thereof. In some embodiments, the invention is an antibody or a polypeptide comprising a fragment or a region of the humanized antibody. In some embodiments, the fragment contains one or more variable regions from a heavy chain and/or a light chain of the humanized antibody as set forth in SEQ ID NO: 1 and SEQ ID NO:2. In yet another embodiment, the fragment contains one or more CDRs from a heavy chain and/or a light chain of the humanized antibody as set forth in SEQ ID NO:1 and SEQ ID NO:2. The invention also provides antibodies that competitively inhibits the immunospecific binding of the humanized antibody to a SM5-1 target antigen.

In some embodiments described herein, the antibody of the invention is a polyclonal antibody, a monoclonal antibody, a Fab fragment, a Fab′ fragment, a F(ab′)₂ fragment, a Fv fragment, a diabody, a single-chain antibody, or a multi-specific antibody formed from antibody fragments described herein.

In another aspect, the invention also provides isolated nucleic acid comprising a nucleotide sequence encoding the heavy chain and/or the light chain, or a fragment thereof, of any of the antibody described herein. In some embodiments, the nucleic acid comprises the nucleotide sequence encoding amino acid sequence set forth in SEQ ID NO:9 and/or SEQ ID NO:10. In some embodiments, the nucleic acid comprises the nucleotide sequence set forth in SEQ ID NO:11 and/or SEQ ID NO:12. In other embodiments, the nucleic acid comprises the nucleotide sequence encoding amino acid sequence set forth in SEQ ID NO:3 and/or SEQ ID NO:4. In other embodiments, the nucleic acid comprises the nucleotide sequence set forth in SEQ ID NO:7 and/or SEQ ID NO:8. In other embodiments, the nucleic acid comprises the nucleotide sequence encoding amino acid sequence set forth in SEQ ID NO: 1 and/or SEQ ID NO:2. In other embodiments, the nucleic acid comprises the nucleotide sequence set forth in SEQ ID NO:5 and/or SEQ ID NO:6.

In another aspect, the invention provides an isolated nucleic acid comprising a nucleotide sequence complementary to any of the nucleotide sequence described herein.

In another aspect, the invention provides a vector containing any of the nucleic acid described herein. The vector may further comprises expression modulation sequence operatively linked to the nucleic acid encoding any of the antibody described herein.

In another aspect, the invention also provides a recombinant cell containing any of the nucleic acid described herein. In some embodiments, the recombinant cell is an eukaryote cell. In other embodiments, the recombinant cell is a CHO cell. In some embodiment, the cell has an accession number ______. In another embodiment, the cell has an accession number ______.

In another aspect, the invention is a method of producing any of the antibody described herein, or a fragment thereof, comprising growing a recombinant cell containing the nucleic acid such that the encoded antibody, or a fragment thereof, is expressed by the cell; and recovering the expressed antibody, or a fragment thereof. In some embodiments, the method further comprises isolating and/or purifying the recovered antibody, or a fragment thereof.

In another aspect, the invention provides a pharmaceutical composition comprising an effective amount of any of the antibody described herein and a pharmaceutically acceptable carrier or excipient. In some embodiments, the pharmaceutical composition comprising an effective amount of a human SM5-1 specific monoclonal antibody and a pharmaceutically acceptable carrier or excipient, wherein the variable region of heavy chain of the human SM5-1 specific monoclonal antibody comprises the amino acid sequences 31-35, 50-66 and 99-108 set forth in SEQ ID NO:9 and the variable region of light chain of the human SM5-1 specific monoclonal antibody comprises the amino acid sequences 24-40, 56-62 and 95-102 set forth in SEQ ID NO:10. In other embodiments, the pharmaceutical composition comprising an effective amount of a humanized SM5-1 specific monoclonal antibody and a pharmaceutically acceptable carrier or excipient, wherein the variable region of heavy chain of the humanized SM5-1 specific monoclonal antibody comprises the amino acid sequences 31-35, 50-66 and 99-108 set forth in SEQ ID NO: 1 and the variable region of light chain of the humanized SM5-1 specific monoclonal antibody comprises the amino acid sequences 24-40, 56-62 and 95-102 set forth in SEQ ID NO:2.

The invention also provides a kit comprising an effective amount of any of the antibody described herein, and an instruction means for administering the antibody.

In another aspect, the invention provides a method for treating neoplasm in a mammal, which method comprises administering to a mammal to which such treatment is needed or desirable, an effective amount of any of the antibody described herein. In some embodiments, the mammal is a human. In some embodiments, the neoplasm is melanoma, breast cancer or hepatocellular carcinoma. In some embodiments, the antibody exerts its anti-neoplasm effect via antibody dependent cell mediated cytotoxicity (ADCC) or complement dependent cell mediated cytotoxicity (CDC). In some embodiments, the antibody is a human antibody. In other embodiments, the antibody is a humanized antibody.

In another aspect, the invention provides a combination, which combination comprises: a) an effective amount of an antibody described herein; and b) an effective amount of an anti-neoplasm agent. In some embodiments, the anti-neoplasm agent is an agent that treats melanoma, breast cancer or hepatocellular carcinoma.

The invention also provides a method for treating neoplasm in a mammal, which method comprises administering to a mammal to which such treatment is needed or desirable, an effective amount of a combination described herein.

In another aspect, the invention provides a method for inducing caspase-10 mediated apoptosis in a cell, which method comprises administering to a cell to which such induction is needed or desirable, an effective amount of any of the antibody described herein. In some embodiments, the cell is a mammalian cell. In some embodiments, the cell is contained in a mammal.

In another aspect, the invention also provides a conjugate, which conjugate comprises any of the antibody described herein conjugated to a toxin and/or a radioactive isotope.

In another aspect, the invention provides a method for assaying for SM5-1 target antigen (e.g., human SM5-1 target antigen) in a sample, which method comprises: a) obtaining a sample from a subject to be tested; b) contacting said sample with any of the antibody described herein under suitable conditions to allow binding between said SM5-1 target antigen, if present in said sample, to said antibody; and c) assessing binding between said SM5-1 target antigen, if present in said sample, to said antibody to determine presence, absence and/or amount of said SM5-1 target antigen in said sample. In some embodiments, the method is used in the prognosis or diagnosis of a neoplasm. In some embodiments, the neoplasm is melanoma, breast cancer or hepatocellular carcinoma.

The invention also provides a kit for assaying for SM5-1 target antigen (e.g., human SM5-1 target antigen) in a sample, which method comprises: a) any of the antibody described herein; and b) means for assessing binding between said SM5-1 target antigen, if present in said sample, to said antibody to determine presence, absence and/or amount of said SM5-1 target antigen in said sample.

### BRIEF DESCRIPTION OF THE DRAWING(S)

FIG. 1 illustrates the expression vector pMG18-3K. Regions of the vector encoding different functions are indicated. HCMV pro, human cytomegalovirus Major Immediate Early promoter; C_{κ}, the human κ chain constant region gene; CH, the human γ1 chain constant region gene; pA, polyadenylation signal; DHFR, dihydrofolate reductase gene; pUC origin, plasmid origin of replication; Amp designates the β-lactamase gene.

FIG. 2 illustrates amino acid sequences of the heavy and light chain variable regions of the humanized anti-SM5-1 antibody (ReSM5-1). The V_{H} of human antibody KOL was chosen as framework for the humanized heavy chain and the V_{L} of human Bence-Jones protein REI was chosen for the humanized light chain. The dashes represent amino acids that are the same as the corresponding residues in human antibodies KOL or REI. The CDRs are enclosed in brackets. Amino acids (in one-letter notation) are numbered according to Kabat (Kabat et al., “Sequences of Proteins of Immunological Interest, 5th ed., US Department of Health and Human Services, National Institute of Health, Bethesda. 1991).

FIG. 3 illustrates the FACS graph showing human anti-SM5-1 antibody (huSM5-1) binding to breast cancer cell lines and melanoma cell lines.

FIG. 4 illustrates the immunofootprinting with human SM5-1 antigen.

FIG. 5 illustrates the changes of activity of caspase-10 in human anti-SM5-1 antibody (huSM5-1) treated QYC and XJC cells.

FIG. 6 illustrates the inhibition of proliferation/growth curve lines for QYC cells treated with humanized and chimeric anti-SM5-1 antibodies. A: growth inhibition curve lines for chSM5-1 treated cells; B: growth inhibition curve lines for ReSM5-1 treated cells.

FIG. 7 illustrates anti-neoplasm effect by chSM5-1 antibody and humanized anti-SM5-1 antibody (ReSM5-1) on QYC cells via ADCC.

FIG. 8 illustrates anti-neoplasm effect by chSM5-1 antibody (A) and humanized anti-SM5-1 antibody (ReSM5-1) (B) on QYC cells via CDC.

FIG. 9 illustrates the therapeutic effect of chSM5-1 and ReSM5-1 for QYC bearing nude mice.

FIG. 10 illustrates the distribution of ¹²⁵I labeled ReSM5-1 and chSM5-1.

### DETAILED DESCRIPTION OF THE INVENTION

The present invention is based on a discovery of a new antigen, SM5-1 antigen, which is over expressed in melanoma, breast cancer and hepatocellular carcinoma. The antigen is present in glycosylated and non-glycosylated form. On Western blot, the antibodies of the invention specifically bound to two proteins having a molecular weight of 230 kD (designated as A230) and 180 kD (designated as A180).

The invention also provides antibodies specific for SM5-1 antigen such as the human antibody (huSM5-1, the variable regions shown in Table 1), the humanized antibody (ReSM5-1, the variable regions are shown in Table 3), and the chimeric antibody (chSM5-1, the variable regions are shown in Table 2). These antibodies can bind to the antigen present in melanoma, breast cancer and hepatocellular carcinoma. These antibodies also inhibit growth and/or proliferation, induce caspase-10 mediated apoptosis in these cancer cells. Thus, SM5-1 antigen provides a target for treating these malignancies.

For clarity of disclosure, and not by way of limitation, the detailed description of the invention is divided into the subsections that follow.

A. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E; Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

B. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), a diabody, a multi-specific antibody formed from antibody fragments, mutants thereof, fusion proteins comprising an antibody portion, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Chothia et al. (1989) Nature 342:877; Al-lazikani et al. (1997) J. Molec. Biol. 273:927-948). As used herein, a CDR may refer to CDRs defined by either approach or by a combination of both approaches.

A “constant region” of an antibody refers to the constant region of the antibody light chain or the constant region of the antibody heavy chain, either alone or in combination.

“Humanized” antibodies refer to a molecule having an antigen binding site substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions; e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody, which are also termed one or more CDRs “derived from” one or more CDRs of the original antibody.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) cancerous cells, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

An “effective amount” of an antibody, drug, or pharmaceutical composition is an amount sufficient to effect beneficial or desired results including clinical results such as shrinking the size of the tumor (in the cancer context, for example, breast or liver cancer), retardation of cancerous cell growth, decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival of individuals. An effective amount can be administered in one or more administrations. For purposes of this invention, an effective amount of drug, compound, or pharmaceutical composition is an amount sufficient to reduce the proliferation of (or destroy) cancerous cells and to reduce and/or delay the development, or growth, of metastases of cancerous cells, either directly or indirectly. As is understood in the cancer clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

C. Compositions and Methods of Making the Compositions

In one aspect, the present invention provides antibodies or polypeptides that specifically bind to SM5-1 antigen. In some embodiments, the antibody is a monoclonal antibody. In some embodiments, the antibody is a human, a humanized, or a chimeric antibody.

In some embodiments, the invention is an antibody or a polypeptide that specifically binds to SM5-1 antigen, wherein the variable region of heavy chain of the antibody comprises the amino acid sequences 31-35, 50-66 and 99-108 set forth in SEQ ID NO:9, and/or the variable region of light chain of the antibody comprises the amino acid sequences 24-40, 56-62 and 95-102 set forth in SEQ ID NO:10. In some embodiments, the heavy chain variable region of the antibody comprises the amino acid sequence set forth in SEQ ID NO:9. In other embodiments, the light chain variable region of the antibody comprises the amino acid sequence set forth in SEQ ID NO: 10. In other embodiments, the heavy chain variable region of the antibody comprises the amino acid sequence set forth in SEQ ID NO:9, and the light chain variable region of the antibody comprises the amino acid sequence set forth in SEQ ID NO: 10. In some embodiments, the antibody is huSM5-1 shown in Table 1.

In some embodiments, the invention is an antibody (e.g., a monoclonal antibody) or a polypeptide, wherein the heavy chain variable region of the antibody comprises the amino acid sequences 31-35, 50-66 and 99-108 set forth in SEQ ID NO:3, and/or the light chain variable region of the antibody comprises the amino acid sequences 24-40, 56-62 and 95-102 set forth in SEQ ID NO:4. In some embodiments, the heavy chain variable region of the antibody comprises the amino acid sequence set forth in SEQ ID NO:3. In some embodiments, the light chain variable region of the antibody comprises the amino acid sequence set forth in SEQ ID NO:4. In some embodiments, the heavy chain variable region of the antibody comprises the amino acid sequence set forth in SEQ ID NO:3, and the light chain variable region of the antibody comprises the amino acid sequence set forth in SEQ ID NO:4. In some embodiments, the antibody is a humanized antibody. In some embodiments, the heavy chain variable region of the humanized antibody comprises the amino acid sequence set forth in SEQ ID NO: 1, and the light chain variable region of the humanized antibody comprises the amino acid sequence set forth in SEQ ID NO:2.

In some embodiments, the antibody is a chimeric antibody, wherein the variable region of heavy chain of said chimeric antibody comprises the amino acid sequences set forth in SEQ ID NO:3. In other embodiments, the antibody is a chimeric antibody, wherein the variable region of light chain of said chimeric antibody comprises the amino acid sequences set forth in SEQ ID NO:4. In other embodiments, the antibody is a chimeric antibody, wherein the variable region of heavy chain of said chimeric antibody comprises the amino acid sequences set forth in SEQ ID NO:3 and the variable region of light chain of said chimeric antibody comprises the amino acid sequences set forth in SEQ ID NO:4.

The invention also provides antibodies (e.g., a monoclonal antibody) and polypeptides that competitively inhibit the immunospecific binding of any of SM5-1 specific monoclonal antibody described herein to a SM5-1 target antigen. Competition assays can be used to determine whether two antibodies bind the same epitope by recognizing identical or sterically overlapping epitopes. Competition assays are known in the art. Typically, antigen is immobilized on a multi-well plate and the ability of unlabeled antibodies to block the binding of labeled antibodies is measured. Common labels for such competition assays are radioactive labels or enzyme labels.

The present invention also encompasses various formulations of the antibodies describe above and equivalent antibodies or polypeptide fragments (e.g., Fab, Fab′, F(ab′)₂, Fv, Fc, etc.), single chain (ScFv), a diabody, a multi-specific antibody formed from antibody fragments, mutants thereof, fusion proteins comprising an antibody portion, and any other modified configuration of the antibodies that comprises a SM5-1 antigen recognition site of the required specificity.

The host cell that produces the human antibody (huSM5-1) having sequences of the variable regions shown in Table 1 is deposited at ______ having accessing number ______ on ______. The host cell that produces the humanized antibody (ReSM5-1) having sequences of the variable regions shown in Table 3 is deposited at ______ having accessing number ______ on ______. This deposit was made under the provisions of the Budapest Treaty on the International Recognition of the Deposit of Microorganisms for the Purpose of Patent Procedure and the Regulations thereunder. Accordingly, the invention provides antibodies, antibody fragments, and polypeptides derived from antibodies produced by the host cells described herein.

The invention also provides any of the following, or compositions (including pharmaceutical compositions) comprising any of the following: (a) antibody huSM5-1 (variable regions shown in Table 1, or produced by the host cell having an accessing number ______ or progeny thereof); (b) a fragment or a region of the antibody huSM5-1; (c) a heavy chain of the antibody huSM5-1; (d) a light chain of the antibody huSM5-1; (e) one or more variable region(s) from a light chain and/or a heavy chain of the antibody huSM5-1; (f) one or more CDR(s) (one, two, three, four, five or six CDRs) of antibody huSM5-1; (g) three CDRs from the light chain of antibody huSM5-1; (h) three CDRs from the heavy chain of antibody huSM5-1; (i) three CDRs from the light chain and three CDRs from the heavy chain, of antibody huSM5-1; and (j) an antibody comprising any one of (b) through (i).

The invention also provides any of the following, or compositions (including pharmaceutical compositions) comprising any of the following: (a) antibody mSM5-1 (variable regions are shown in Table 2, or produced by the host cell having an ATCC Designition No. HB-12588 or progeny thereof); (b) a fragment or a region of the antibody mSM5-1; (c) one or more variable region(s) from a light chain and/or a heavy chain of the antibody mSM5-1; (d) one or more CDR(s) (one, two, three, four, five or six CDRs) of antibody mSM5-1; (e) three CDRs from the light chain of antibody mSM5-1; (f) three CDRs from the heavy chain of antibody mSM5-1; (g) three CDRs from the light chain and three CDRs from the heavy chain, of antibody mSM5-1; and (h) an antibody comprising any one of (b) through (g).

The invention also provides any of the following, or compositions (including pharmaceutical compositions) comprising any of the following: (a) antibody ReSM5-1 (variable regions are shown in Table 3, or produced by the host cell having an accessing number ______ or progeny thereof); (b) a fragment or a region of the antibody ReSM5-1; (c) one or more variable region(s) from a light chain and/or a heavy chain of the antibody ReSM5-1; (d) one or more CDR(s) (one, two, three, four, five or six CDRs) of antibody ReSM5-1; (e) three CDRs from the light chain of antibody ReSM5-1; (f) three CDRs from the heavy chain of antibody ReSM5-1; (g) three CDRs from the light chain and three CDRs from the heavy chain, of antibody ReSM5-1; and (h) an antibody comprising any one of (b) through (g).

It is understood that in some embodiments, the CDR can be a Kabat CDR or a Chothia CDR or a combination of the Kabat and Chothia CDR. Determination of CDR regions is well within the skill of the art.

In some embodiments, the invention provides an antibody which comprises at least one CDR that is substantially homologous to at least one CDR, at least two, at least three, at least four, at least five CDRs of huSM5-1, mSM5-1, or ReSM5-1 (or, in some embodiments substantially homologous to all 6 CDRs of huSM5-1, mSM5-1, or ReSM5-1, or derived from huSM5-1, mSM5-1, or ReSM5-1). Other embodiments include antibodies which have at least two, three, four, five, or six CDR(s) that are substantially homologous to at least two, three, four, five or six CDRs of huSM5-1, mSM5-1, or ReSM5-1, or derived from huSM5-1, mSM5-1, or ReSM5-1. It is understood that, for purposes of this invention, binding specificity and/or overall activity (which may be in terms of treating cancer (e.g., melanoma, breast cancer, and hepatocellular carcinoma) is generally retained, although the extent of activity may vary compared to huSM5-1, mSM5-1, or ReSM5-1 (may be greater or lesser)).

The invention also provides a polypeptide (which may or may not be an antibody) which comprises an amino acid sequence that has any of the following: at least 5 contiguous amino acids, at least 8 contiguous amino acids, at least about 10 contiguous amino acids, at least about 15 contiguous amino acids, at least about 20 contiguous amino acids, at least about 25 contiguous amino acids, at least about 30 contiguous amino acids of a variable sequence of the antibody described herein (such huSM5-1, mSM5-1, and ReSM5-1).

The invention also provides methods of making any of these antibodies or polypeptides. The antibodies of this invention can be made by procedures known in the art, some of which are illustrated in the Examples. In some embodiments, the method comprises growing a recombinant cell containing the nucleic acid encoding any of the antibody described herein or a fragment thereof (such as nucleic acid encoding huSM5-1 shown in Table 1, and ReSM5-1 shown in Table 3) such that the encoded antibody or a fragment thereof is expressed, and recovering the expressed antibody or a fragment thereof. In some embodiments, the method further comprises isolating and/or purifying the recovered antibody or a fragment thereof.

The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available.

Monoclonal antibodies may be prepared using hybridoma methods, such as those described by Kohler and Milstein, 1975, Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The antibodies or fragments of the invention may also be made by recombinant DNA methods, such as those described in U.S. Pat. No. 4,816,567. DNA encoding the antibodies or fragments is isolated and sequenced using conventional procedures, such as by using oligonucleotide probes that are capable of binding specifically to genes encoding the heavy and light chains of the antibodies. Once isolated, the DNA (for example, SEQ ID NO:5 and SEQ ID NO:6) may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the antibodies in the recombinant host cells. Vectors (including expression vectors) and host cells are further described herein.

The invention includes modifications to antibodies described herein, including functionally equivalent antibodies which do not significantly affect their properties and variants which have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tyrosine. These polypeptides also include glycosylated and nonglycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the variable region. Changes in the variable region can alter binding affinity and/or specificity. Other methods of modification include using coupling techniques known in the art, including, but not limited to, enzymatic means, oxidative substitution and chelation. Modifications can be used, for example, for attachment of labels for immunoassay.

The invention also encompasses fusion proteins comprising one or more fragments or regions from the antibodies of this invention. In one embodiment, the fusion polypeptide contains a light chain variable region and/or a heavy chain variable region set forth in SEQ ID NO:2 and/or SEQ ID NO: 1. In other embodiments, the fusion polypeptide contains a light chain variable region and/or a heavy chain variable region set forth in SEQ ID NO:10 and/or SEQ ID NO:9. For purposes of this invention, a fusion protein contains one or more antibodies and another amino acid sequence to which it is not attached in the native molecule, for example, a heterologous sequence or a homologous sequence from another region. Exemplary heterologous sequences include, but are not limited to a “tag” such as a FLAG tag or a 6His tag. Tags are well known in the art. The antibodies or fragments thereof disclosed herein may be used to make anti-tumor bifunctional fusion proteins, such as chimeric proteins as described in co-pending U.S. application Ser. No. ______ (Attorney Docket No. 54906-2000200; Title: Preparation and application of anti-tumor bifunctional fusion proteins) filed Nov. 26, 2003, which is incorporated in its entirety by reference.

A fusion polypeptide can be created by methods known in the art, for example, synthetically or recombinantly. Typically, the fusion proteins of this invention are made by preparing an expressing a polynucleotide encoding them using recombinant methods described herein, although they may also be prepared by other means known in the art, including, for example, chemical synthesis.

In another embodiment, the chimeric antibody of the invention are provided in which the heavy and/or light chains are fusion proteins. In some embodiments, the constant domain of the chains is from one particular species and/or class, and the variable domains are from a different species and/or class. For instance, a chimeric antibody (in some embodiments) is one in which the constant regions are derived from human origin, and the variable regions are homologous or derived from a murine antibody (for example, SEQ ID NO:3 and SEQ ID NO:4). Also embodied within the invention is an antibody with a humanized variable region, in which (in some embodiments) the CDR regions comprise murine amino acid sequences, while the framework regions are derived from human sequences. Other forms of humanized antibodies are known in the art and described herein. Also embodied are functional fragments of chimeras. An example is a humanized Fab fragment, which contains a human hinge region, a human first constant region, a human kappa light or heavy chain constant region, and the variable region of light and/or heavy chain from a mouse antibody (for example, SEQ ID NO:3 and SEQ ID NO:4). The humanized Fab fragments can in turn be made to form Fab dimers. Typically, the fusion proteins and chimeras of this invention are made by preparing an expressing a polynucleotide encoding them using recombinant methods described herein, although they may also be prepared by other means known in the art, including, for example, chemical synthesis. See, for example, U.S. Pat. Nos. 5,807,715; 4,816,567; and 6,331,415.

The invention also encompasses humanized antibodies. The polynucleotide sequence of an antibody (for example, SEQ ID NO:7 and SEQ ID NO:8) or other equivalent antibodies may be used for genetic manipulation to generate a “humanized” antibody, or to improve the affinity, or other characteristics of the antibody. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. For example, the constant region may be engineered to more resemble human constant regions to avoid immune response if the antibody is used in clinical trials and treatments in humans. See, for example, U.S. Pat. Nos. 5,997,867 and 5,866,692.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J Immunol. 138:4534-4538 (1987), and Brown et al. Cancer Res. 47:3577-3583 (1987). Other references describe rodent CDRs grafted into a human supporting framework region (FR) prior to fusion with an appropriate human antibody constant domain. See, for example, Riechmann et al. Nature 332:323-327 (1988), Verhoeyen et al. Science 239:1534-1536 (1988), and Jones et al. Nature 321:522-525 (1986). Another reference describes rodent CDRs supported by recombinantly veneered rodent framework regions. See, for example, European Patent Publication No. 519,596. These “humanized” molecules are designed to minimize unwanted immunological response toward rodent anti-human antibody molecules which limits the duration and effectiveness of therapeutic applications of those moieties in human recipients. Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; 6,350,861; and PCT WO 01/27160.

In another alternative, antibodies may be made recombinantly by phage display technology. See, for example, U.S. Pat. Nos. 5,565,332; 5,580,717; 5,733,743 and 6,265,150; and Winter et al., Annu. Rev. Immunol. 12:433-455 (1994), and Example 2. Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT patent application PCT WO 9306213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

This invention also provides antibodies or polypeptides described herein conjugated (for example, linked) to a therapeutic agent, such as a radioactive moiety, a toxin (e.g., calicheamicin), or a chemotherapeutic molecule, a prodrug-activating enzyme which converts a prodrug to an active anti-cancer drug, or to liposomes or other vesicles containing chemotherapeutic compounds (or compositions comprising these antibodies or polypeptides). The compositions, when administered to an individual, can target these agents to a cancer cell expressing SM5-1 antigen recognized by the antibody or polypeptide(s) and thus can, for example, eliminate (or reduce the number of) cancerous cells and/or suppress proliferation and/or growth of cancerous cells. These, conjugation generally refers to linking these components as described herein. The linking (which is generally fixing these components in proximate association at least for administration) can be achieved in any number of ways, as described below.

A radioactive moiety or molecule of this invention includes any radioisotope which is effective in destroying a cancerous cell. Examples include, but not limited to, cobalt-60, ¹³¹I, and X-rays. Additionally, naturally occurring radioactive elements such as uranium, radium, and thorium which typically represent mixtures of radioisotopes, are suitable examples of a radioactive molecule.

A toxin of the invention include, but not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The antibodies or polypeptides of the invention can be conjugated (linked) to a radioactive moiety or molecule, a toxin, or other therapeutic agents, a prodrug-activating enzyme which converts a prodrug to an active anti-cancer drug, or to liposomes or other vesicles containing therapeutic agents covalently or non-covalently, directly or indirectly. The antibody may be linked to the radioactive molecule, the toxin, the therapeutic molecule, or a prodrug-activating enzyme at any location along the antibody so long as the antibody is able to bind its target antigen.

A toxin or a therapeutic agent may be coupled (e.g., covalently bonded) to a suitable monoclonal antibody either directly or indirectly (e.g., via a linker group, or, alternatively, via a linking molecule with appropriate attachment sites, such as a platform molecule as described in U.S. Pat. No. 5,552,391). The toxin and therapeutic agent of the present invention can be coupled directly to the particular targeting proteins using methods known in the art. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibody or polypeptide conjugates of the present invention may include a bifunctional linker which contains both a group capable of coupling to a toxic agent or therapeutic agent and a group capable of coupling to the antibody. A linker can function as a spacer to distance an antibody from an agent in order to avoid interference with binding capabilities. A linker can be cleavable or non-cleavable. A linker can also serve to increase the chemical reactivity of a substituent on an agent or an antibody, and thus increase the coupling efficiency. An increase in chemical reactivity may also facilitate the use of agents, or functional groups on agents, which otherwise would not be possible. The bifunctional linker can be coupled to the antibody by means which are known in the art. For example, a linker containing an active ester moiety, such as an N-hydroxysuccinimide ester, can be used for coupling to lysine residues in the antibody via an amide linkage. In another example, a linker containing a nucleophilic amine or hydrazine residue can be coupled to aldehyde groups produced by glycolytic oxidation of antibody carbohydrate residues. In addition to these direct methods of coupling, the linker can be indirectly coupled to the antibody by means of an intermediate carrier such as an aminodextran. In these embodiments the modified linkage is via either lysine, carbohydrate, or an intermediate carrier. In one embodiment, the linker is coupled site-selectively to free thiol residues in the protein. Moieties which are suitable for selective coupling to thiol groups on proteins are well known in the art. Examples include disulfide compounds, α-halocarbonyl and α-halocarboxyl compounds, and maleimides. When a nucleophilic amine function is present in the same molecule as an α-halo carbonyl or carboxyl group the potential exists for cyclization to occur via intramolecular alkylation of the amine. Methods to prevent this problem are well known to one of ordinary skill in the art, for example by preparation of molecules in which the amine and α-halo functions are separated by inflexible groups, such as aryl groups or trans-alkenes, that make the undesired cyclization stereochemically disfavored. See, for example, U.S. Pat. No. 6,441,163 for preparation of conjugates of maytansinoids and antibody via a disulfide moiety.

An antibody (or polypeptide) of this invention may be conjugated (linked) to a radioactive moiety or molecule by any method known to the art. For a discussion of methods for radiolabeling antibody see “Cancer Therapy with Monoclonal AntibodiesT”, D. M. Goldenberg ed. (CRC Press, Boca Raton, 1995).

The antibodies (or polypeptides) of the invention may be linked to an agent (including a prodrug-activating enzyme) which converts a prodrug to an active anti-cancer drug. For example, the antibodies (or polypeptides) of this invention may be used in Antibody Dependent Enzyme Mediated Prodrug Therapy (ADEPT) by conjugating (linking) the antibody to a prodrug-activating enzyme which converts a prodrug (e.g., a peptidyl chemotherapeutic agent, see WO81/01145) to an active-cancer drug. See, for example, WO 88/07378 and U.S. Pat. No. 4,975,278.

An antibody (or polypeptide) of this invention may be linked to a labeling agent (alternatively termed “label”) such as a fluorescent molecule, a radioactive molecule or any others labels known in the art. Labels are known in the art which generally provide (either directly or indirectly) a signal.

This invention encompasses compositions, including pharmaceutical compositions, comprising effective amount of antibodies (including antibody conjugates) and polypeptides that bind to SM5-1 antigen, and polynucleotides comprising sequences encoding antibodies, polypeptides described herein. As used herein, compositions comprise one or more antibodies that bind to SM5-1 antigen, and/or one or more polynucleotides comprising sequences encoding one or more antibodies that bind to SM5-1. These compositions may further comprise suitable excipients, such as pharmaceutically acceptable excipients including buffers, which are well known in the art.

This invention also encompasses a combination comprising an effective amount of any of the antibodies described herein and an effective amount of an anti-neoplasm agent. The anti-neoplasm agent can be an agent that treats melanoma, breast cancer, or hepatocellular carcinoma.

The invention also provides an isolated SM5-1 target antigen, which comprises a protein that specifically binds to the antibodies described herein. In some embodiments, the isolated SM5-1 antigen is a human antigen. In some embodiments, the isolated SM5-1 antigen is a glycosylated protein. In other embodiments, the isolated SM5-1 antigen is a non-glycosylated protein. In some embodiments, the isolated SM5-1 antigen is a fragment of A230 or A180 described herein. In some embodiments, the isolated SM5-1 antigen is isolated from a melanoma, breast cancer and/or hepatocellular carcinoma cell.

D. Polynucleotides Vectors and Host Cells

The invention also provides isolated polynucleotides encoding the antibodies of the invention (for example, an antibody comprising the polypeptide sequences of the light chain and heavy chain variable regions set forth in SEQ ID NO:2 and SEQ ID NO: 1, and an antibody comprising the polypeptide sequences of the light chain and heavy chain variable regions set forth in SEQ ID NO:10 and SEQ ID NO:9), and vectors and host cells comprising the polynucleotide.

In another aspect, the invention provides polynucleotides encoding any of the polypeptides (including antibody fragments) described herein.

In another aspect, the invention provides compositions (such as a pharmaceutical compositions) comprising any of the polynucleotides of the invention. In some embodiments, the polynucleotides comprises nucleotide sequences set forth in SEQ ID NO: 11 or SEQ ID NO: 12. In some embodiments, the polynucleotides comprises nucleotide sequences set forth in SEQ ID NO:5 or SEQ ID NO:6. In some embodiments, the composition comprises an expression vector comprising a polynucleotide encoding the antibody as described herein. In still other embodiments, the composition comprises either or both of the polynucleotides described herein. Expression vectors, and administration of polynucleotide compositions are further described herein.

In another aspect, the invention provides a method of making any of the polynucleotides described herein.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably at least about 80% identity and most preferably at least about 90% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D. C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2× SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC (sodium chloride/sodium citrate) and 50% formamide at 55° C., followed by a high-stringency wash consisting of 0.1× SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston (1994).

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., (1989), for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, pUC57, pMG18-3K, Bluescript (e.g., pBS SK+, pBS SK−) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, and cosmids. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. Suitable non-mammalian host cells include prokaryotes (such as E. coli or B. subtillis) and yeast (such as S. cerevisae, S. pombe; or K. lactis). Preferably, the host cells express the cDNAs at a level of about 5 fold higher, more preferably 10 fold higher, even more preferably 20 fold higher than that of the corresponding endogenous antibody or protein of interest, if present, in the host cells. Screening the host cells for a specific antibody binding to SM5-1 target antigen is effected by an immunoassay or FACS. A cell overexpressing the antibody or protein of interest can be identified.

E. Methods of Diagnosing Cancer Using Antibodies that Specifically Bind to SM5-1 Antigen

In one aspect, the invention provides methods for assaying for human SM5-1 target antigen in a sample, which method comprises: a) obtaining a sample from a subject to be tested; b) contacting said sample with an antibody specific for SM5-1 target antigen; and c) assessing binding between said human SM5-1 target antigen, if present in said sample, to said antibody to determine presence, absence and/or amount of said human SM5-1 target antigen in said sample.

Antibodies specific for SM5-1 target antigen described herein may be used to identify the presence or absence of cancerous cells, including but not limited to, melanoma, breast cancer, and hepatocellular carcinoma for purposes of diagnosis. Detection generally involves contacting cells with an antibody specific for SM5-1 target antigen described herein that binds to the antigen and the formation of a complex between the antigen and the antibody. The formation of such a complex can be in vitro or in vivo.

In another aspect, the invention provides methods of aiding diagnosis of cancer using any antibodies or polypeptides described herein. As used herein, methods for “aiding diagnosis” means that these methods assist in making a clinical determination regarding the classification, or nature, of cancer, and may or may not be conclusive with respect to the definitive diagnosis. Accordingly, a method of aiding diagnosis of cancer can comprise the step of detecting the level of SM5-1 target antigen in a biological sample from the individual and/or determining the level of SM5-1 target antigen expression in the sample.

One method of using the antibodies for diagnosis is in vivo tumor imaging by linking the antibody to a labeling moiety (e.g., a fluorescent agent, a radioactive or radioopaque agent), administering the antibody to the individual and using an x-ray or other imaging machine to visualize the localization of the labeled antibody at the surface of cancer cells expressing the antigen. The antibody is administered at a concentration that promotes binding at physiological conditions. Labeling moieties are known in the art.

In other methods, the cancerous cells are removed and the tissue prepared for immunohistochemistry by methods well known in the art (e.g., embedding in a freezing compound, freezing and sectioning, with or without fixation; fixation and paraffin embedding with or without various methods of antigen retrieval and counterstaining). The antibodies may also be used to identify cancerous cells at different stages of development. The antibodies may also be used to determine which individuals' tumors express the antigen on their surface at a pre-determined level and are thus candidates for immunotherapy using antibodies directed against said antigen.

Antibodies (or polypeptides) recognizing the antigen may also be used to create diagnostic immunoassays for detecting antigen released or secreted from living or dying cancer cells in bodily fluids, including but not limited to, blood, saliva, urine, pulmonary fluid, or ascites fluid. Methods of using antibodies of the invention for diagnostic purposes is useful both before and after any form of anti-cancer treatment, e.g., chemotherapy or radiation therapy, to determine which tumors are most likely to respond to a given treatment, prognosis for individual with cancer, tumor subtype or origin of metastatic disease, and progression of the disease or response to treatment.

F. Methods of Using Antibodies Specific for SM5-1 Antigen for Therapeutic Purposes

The present invention also provides a method for treating neoplasm in a mammal, which method comprises administering to a mammal to which such treatment is needed or desirable, an effective amount of an antibody specific to SM5-1 antigen described herein (for example, huSM5-1 and ReSM5-1). The antibodies described in this invention may be used for therapeutic purposes in individuals with cancer in a variety of tissues, including but not limited to, melanoma, breast cancer, and hepatocellular carcinoma. In some embodiments, the antibody is used for passive immunity of cancer patients. In some embodiments, the antibody administered exerts its anti-neoplasm effect via antibody dependent cell mediated cytotoxicity (ADCC) and/or complement dependent cell mediated cytotoxicity (CDC). In some embodiments, the antibody is used for treating neoplasm in human.

The invention also provides a method for treating neoplasm in a mammal comprising administering to a mammal to which such treatment is needed or desirable, an effective amount of a combination which comprises an effective amount of an antibody specific to SM5-1 described herein and an effective amount of an anti-neoplasm agent. In some embodiments, the anti-neoplasm agent is an agent that treats melanoma, breast cancer, or hepatocellular carcinoma.

The invention also provides a method for inducing caspase-10 mediated apoptosis in cell, which method comprises administering to a cell to which such induction is needed or desirable, an effective amount of an antibody specific to SM5-1 described herein. In some embodiments, the cell is a mammalian cell. In some embodiments, the cell is contained in a mammal.

Various formulations of anti-SM5-1 antibodies described herein and equivalent antibodies or fragments (e.g., Fab, Fab′, F(ab′)₂, Fv, Fc, etc.), such as chimeric antibodies, single chain (ScFv), mutants thereof, fusion proteins comprising an antibody portion, humanized antibodies, antibodies conjugated to a toxin or a radioactive isotope and any other modified configuration that comprises the required specificity, thereof may be used for administration. In some embodiments, the antibodies or various formulations thereof may be administered neat. In other embodiments, the antibodies or various formulations (including any composition embodiment described herein) thereof and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intramuscularly, etc.), although other forms of administration (e.g., oral, mucosal, etc) can be also used. Accordingly, antibody and equivalents thereof are preferably combined with pharmaceutically acceptable vehicles such as saline, Ringer's solution, dextrose solution, and the like. The particular dosage regimen, i.e., dose, timing and repetition, will depend on the particular individual and that individual's medical history. Generally, a dose of at least about 100 ug/kg body weight, at least about 250 ug/kg body weight, at least about 750 ug/kg body weight, at least about 3 mg/kg body weight, at least about 5 mg/kg body weight, at least about 10 mg/kg body weight is administered. For example, a dose of 1-200 mg/per day may be administered. The antibody may be injected into the tumor in situ (Irie et al., Proc. Natl. Acad. Sci. USA 83:8694-8698 (1986)) or administered systematically (especially for metastasis).

Empirical considerations, such as the half-life, generally will contribute to the determination of the dosage. Antibodies which are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is based on reducing the number of cancerous cells, maintaining the reduction of cancerous cells, reducing the proliferation of cancerous cells, or delaying the development of metastasis. The presence of cancerous cells can be identified by any number of methods known to one of skill in the art or discussed herein (e.g., detection by immunohistochemistry or flow cytometry of biopsies or biological samples). Alternatively, sustained continuous release formulations of antibodies may be appropriate. Various formulations and devices for achieving sustained release are known in the art.

In one embodiment, dosages for antibodies may be determined empirically in individuals who have been given one or more administration(s). Individuals are given incremental dosages of the antibodies. To assess efficacy of the antibodies, a marker of the specific cancer disease state can be followed. These include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In some embodiments, more than one antibody or other agent may be present. The antibodies can be monoclonal or polyclonal. Such compositions may contain at least one, at least two, at least three, at least four, at least five different antibodies that are reactive against carcinomas, adenocarcinomas, sarcomas, or adenosarcomas. A mixture of antibodies, as they are often denoted in the art, may be particularly useful in treating a broader range of population of individuals.

G. Kits Comprising Antibodies of the Invention

The invention also provides kits comprising antibodies for use in detection and/or therapy. In some embodiments, the kit comprises any antibodies described herein. The kits of this invention are in suitable packaging, and may optionally provide additional components such as, buffers and instructions for use of the antibody in any of the methods described herein.

In one aspects, the kits may be used for any of the methods described herein, including, for example, to treat an individual with a neoplasm. In some embodiments, the kit comprises an effective amount of an antibody described herein, and an instruction means for administering said antibody.

In another aspect, the invention provides a kit for assaying for human SM5-1 target antigen in a sample, which kit comprises an antibody described herein and means for assessing binding between the human SM5-1 target antigen, if present in the sample, to the antibody to determine presence, absence, and/or amount of the target antigen in the sample. In some embodiments, the kit further comprises an instruction means for performing the assay.

### H. EXAMPLES

### Example 1

### Screening and Identification of Human SM5-1 Antigen

1. Construction of cDNA Library of Hepatocellular Carcinoma Cell Line QYC

Total RNAs were extracted from hepatocellular carcinoma cell line QYC with Trizol reagents. Then mRNAs were isolated and cDNA was synthesized as described (Marken J S. PNAS, 1992, 89:3503-3507). The cDNA was inserted into mammalian transient expression vector pCDM8 (from Invitrogen.) after ligation of the non-self-complementary BstXI adaptors and transformed into the E. coli. MC1061/P3 (from Invitrogen) by electroporation to construct the cDNA library.

2. Expression and Screening of the cDNA Library

COS-7 (Invitrogen) cells were transfected with the above acquired cDNA library using Lipofection method. After twelve hours, the cells were digested and plated in new flasks. Seventy-two hours after transfection, the cells were harvested and re-suspended in PBS/0.5 mM EDTA/5% FBS containing mouse monoclonal antibody specific for SM5-1 antigen (designated as mSM5-1, the hybridoma producing this antibody was deposited in the American Type Culture Collection (ATCC) on Oct. 10, 1998, with a Patent Deposit Designation of HB-12588). After one hour in ice bath, the cells were harvested again, re-suspended in PBS/EDTA/0.5% FBS, and replated on 10 petri dishes pre-coated with goat anti-mouse Ig secondary antibodies. After 2 h at room temperature, the cells were then carefully washed with PBS/EDTA/5% FBS to remove unbound cells. Plasmid DNA was recovered from the adherent cells by Hirt method (Hirt B. J Mol Biol, 1967, 26:365-369). The recovered plasmid DNA was transformed into E. coli MC1061/p3 cells and the transformed E. coli were used to prepare a second cDNA library.

After 4 rounds of transfection, expression, screening and plasmid harvesting described above, the final harvested plasmid acquired with Hirt method was transformed into E. coli. MC1061/P3. Many clones were then randomly selected. Plasmid was extracted from these clones and used to transfect COS-7 cells by Lipofectin method. Twelve hours after transfection, the cells were digested with trypsin and plated on new plastic dishes. Seventy-two hours after transfection, mSM5-1 was added into dish, and stained with FITC-labeled goat anti-mouse Ig secondary antibody. Positive clones were identified under fluorescent microscope. Finally, the plasmid cDNA was isolated from a single postive clone. Then the cDNA clone encoding the SM5-1 antigen was sequenced and analyzed. The extracellur region of SM5-1 antigen was cloned into a mammalian expression vector and the construted vector was transfected into CHO cells for expression. The extracellur region of SM5-1 antigen was purified by affinity chromatography (mouse SM5-1 antibody immobilized on Sepharose-4B) from the serum-free culture supernatant.

### Example 2

### Screening for Variable Region Gene of a Human Anti-Human SM5-1 Antibody from a Human Antibody Library

The human antibody library was constructed according to methods described by Marks et al (J. Mol. Biol. 222, 581-597), Hoogenboom and Winter (J. Mol. Biol, 227, 381-388), Haidais C G et al (J. Immunol. Methods., 2001, Nov. 1; 257(1-2): 185-202), Griffiths, A. D. et al. (EMBO J., 13, 3245-3260(1994)); Nissim, A, et al. (EMBO J, 13, 692-698(1994)).

The recovered antibody library was added into 14 ml fresh LB media and cultured for 16 h in a 50 ml triangle bottle at 37° C.

The bacteria were centrifuged at 12,000 rpm for 10 min. The supernatant was transferred to a sterile 50 ml centrifuge tube and stored for later use and the titer should be higher than 2×10¹¹. Cell culture flask was coated with the purified antigen SM5-1 acquired in Example 1. No less than 3×10¹⁰ phage particles were added into the flask and incubated at 37° C. for 1 h. The flask was washed with 10 ml PBS containing 1% Tween-20 for 10 times. The adherent particles were eluted by elution buffer and added into 1 ml TG1 cells growing in logarithmic stage. The cells were cultured in a shaker at 37° C. for 16 h.

Steps described in the previous paragraph were repeated for another 3 times.

The above acquired cells were diluted into 10⁵/ml, and then cultured on a 0.1% Amp, 1.5% agar plate to acquire single clones. The clones were cultured on a deep-well 96-well plate, one well for each clone; totaling 960 clones (10 plates) were obtained. The plates were centrifuged at 5000 rpm for 20 min, and the supernatant was transferred to a sterile deep-well plate and covered and stored at 4° C.

In ten 96-well plates, each well and wells was coated with 10 μl SM5-1 antigen (10 μg/ml). Supernatant described above (10 μl) was added into each well and the plates were incubated at 37° C. for 1 h. The wells were washed 20 times with PBS containing 1% Tween-20. Then, 1 ul HRP-labeled goat anti-M13 mAb was added into each well and the plates were incubated at 37° C. for 30 min. The wells were washed 10 times with PBS containing 1% Tween-20.

After wash, 100 μl TMB substrate solution were added into the well and the plates were incubated at room temperature without light for 5-20 min to develop the color. And 50 ul stop solution was added into each well. The plates were read at 450 nm.

There were 415 positive clones selected through the process. The higher OD₄₅₀ wells correspond with the clones containing the variable region of the antibodies with higher affinity. According to the optical adsorption, five clones were selected. These 5 clones were seeded into 100 ml LB culture media, and cultured for 9 h at 37° C. in a shaker with 260 rpm. Then IPTG was added into the culture at the final concentration of 1 mM and the culture was incubated for 10 h for induction. Then anti-SM5-1 protein was isolated and purified, which is a human antibody against human SM5-1 antigen. This human antibody (huSM5-1) was purified for affinity determination and the positive clone with the highest affinity was selected for further research. The amino acid sequence and the nucleotide sequence for the heavy chain variable region (SEQ ID NOS: 9 and 11) and the light chain variable region (SEQ ID NOS: 10 and 12) of this antibody are shown in Table 1 below.

TABLE 1

Amino acid sequence and nucleotide sequence
of the heavy chain and light chain variable
region of human anti-SM5-1 antibody (huSM5-1)

| | |
|---|---|
| huSM5-1 heavy chain variable | |
| region amino acid sequence | |
| QVQLVESGGGVVQPGCSLRLSCSSSGYTFTSYTM | (SEQ ID NO: 9) |
| HWVRQAPGKGLEWIGYINPYNDGGKYNEKFKWRF | |
| SISSDKSKNTLFLQSDSLTPEDTGVYYCARGSRY | |
| DWYGDYWGQGTPVTVSS | |
| huSM5-1 light chain variable | |
| region amino acid sequence | |
| DIQMTQSPSSLSGSVGDRVTITCDSSQSVLYSSK | (SEQ ID NO: 10) |
| DDNYLAWYQQGPGKAPSLLIYYASDRESDVPSRF | |
| SGSGSGDDYTLTISSLQPEDAATYYCHQWFSSYT | |
| FDQGTKLNITR | |
| huSM5-1 heavy chain variable | |
| region nucleotide sequence | |
| CAGGTGCAGCTGGTGGAGTCTGGCGGTGGAGTGG | (SEQ ID NO: 11) |
| TCCAGCCCGGCTGCAGCCTGAGGCTGTCCTGCAG | |
| TAGCTCTGGCTACACCTTCACCAGCTACACCATG | |
| ACATGGGTGCGCCAAGCCCCCGGAAAGGGCCTCG | |
| AATGGATTGGCTACATTAATCCTTATAATGACGG | |
| TGGGAAGTACAATGAAAAGTTCAAGTGGAGATTT | |
| TCAATATCAAGTGACAAGAGCAAGAACACCCTGT | |
| TCCTCCAAAGCGACAGCTTGACCCCAGAGGACAC | |
| CGGCGTATACTATTGTGTGCGCGGCAGCCGTTAC | |
| GACTGGTACGGGGACTACTGGGGCCAAGGCACTC | |
| CAGTCACCGTCTCCTCT | |
| huSM5-1 light chain variable | |
| region nucleotide sequence | |
| GACATCCAGATGACTCAGAGCCCATCCAGCTTGA | (SEQ ID NO: 12) |
| GCGGCTCAGTAGGCGACCGCGTAACGATCACTTG | |
| CGACTCCTCTCAGTCAGTATTGTACTCCAGCAAA | |
| GACGACAACTACCTGGCCGGATATCAGCAGGGGC | |
| CCGGCAAAGCCCCAAGCTTGCTGATTTATTATGC | |
| CTCCGACCGCGAGTCTGACGTGCCATCACGCTTT | |
| AGCGGCAGCGGGTCCGGTGATGATTACACGCTGA | |
| CCATTAGCAGTCTGCAGCCTGAGGACGCCGCCAC | |
| CTACTACTGTCACCAGTGGTTTAGTTCCTACACT | |
| TTTGACCAGGGAACTAAACTGAACATTACTCGA | |

### Example 3

### The expression of the Human Antibody Against Human SM5-1 Antigen

1. The Construction of Expression Vector

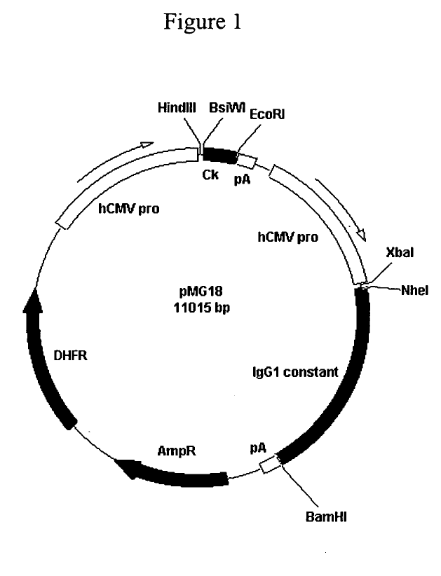

Using PCR method, XbaI site and the signal peptide of mAb OKT3 were added to the 5′end of the heavy chain variable region gene (VH) of huSM5-1 and a NheI site added to the 3′end. The amino acid sequence of mAb OKT3 signal peptide is MDFQVQIFSFLLISASVIISRG (SEQ ID NO: 13), and the nucleotide sequence of mAb OKT3 signal peptide is ATGGATTTTCAGGTGCAGATTTTCAGCTTCCTGCT AATCAGTGCCTCAGTCATAATATCCAGAGGAG (SEQ ID NO:14). The PCR product was cloned into pGEM-T vector and its sequence was verified. The V_{H} was excised by XbaI and NheI digestion and then, inserted into the expression vector pMG18-3K shown in FIG. 1 (from Development of tools for environmental monitoring based on incp-9 plasmid sequences. A. Greated, R. Krasowiak, M. Titok, C. M. Thomas school of biological sciences, university of Bermingham, Edgbaston, Birmingham B 15 2TT, UK and Faculty of Biology, Dept of Microbiology, Belarus State University Scorina Av. 4, Minsk 220080 Belarus) at the position of XbaI/NheI.

Using PCR method, HindIII site and signal peptide of mAb OKT3 were added to the 5′end of the light chain variable region gene (VL) of huSM5-1 and a BsiWI site added to the 3′end. The PCR product was cloned into pGEM-T vector and its sequence was verified. The VL was excised by HindIII and BsiWI digestion and then, inserted into the expression vector pMG18-3K at the position of HindIII/BsiWI.

The expression vector for human antibody against human SM5-1 antigen was constructed.

Prior to transfection, CHOdhfr-cells were maintained in complete DMEM medium containing glycin, hypoxanthine and thymidine (GHT). The expression vector described above was transfected into CHOdhfr-cells using Lipofectamine 2000 reagent (Invitrogen, Garlsbad, Calif.) according to the manufacture's instruction. The transfected cells were then selected in GHT free DMEM medium containing stepwise increments in MTX level up to 1.0 M. Drug resistant clones were picked and expanded for further analysis. The culture supernatants from cell clones were analyzed for antibody production by the sandwich ELISA which used goat anti-human IgG (Fc) (KPL) as capture antibody and goat anti-human kappa-HRP (KPL) as detector antibody. Purified human IgG1/Kappa (Sigma) was used as a standard in the ELISA assay. The clone producing the highest amount of antibody was selected and grown in serum-free medium. The recombinant antibodies were purified by Protein A affinity chromatography from the serum-free culture supernatant.

### Example 4

### Construction of Humanized and Chimeric Antibody of the Mouse Anti-SM5-1 Antibody (mSM5-1)

1. Cloning of Mouse Anti-SM5-1 Antibody Heavy and Light Chain Variable Region Genes.

RNA was isolated from SM5-1 (IgG1, κ) hybridoma cells (ATCC Designation No. HB-12588) with TRIzol Reagent (Gibco BRL, Grand Island, N.Y.). The heavy and light variable region cDNAs of mSM5-1 were cloned from hybridoma cells using 5′RACE system (Gibco BRL, Gaithersburg, Md.) according to the manufacture's instruction. The final PCR products were cloned into pGEM-T vector (Promega, Madison, WI) for sequence determination. The nucleotide sequence and the deduced amino acid sequences of heavy (mSM5-1 VH) and light (mSM5-1 VL) variable region are shown in Table 2 below.

TABLE 2

Nucleotide and amino acid sequences for mouse
anti-SM5-1 antibody (mSM5-1) variable regions

| | |
|---|---|
| mSM5-1 heavy chain variable | |
| region amino acid sequence | |
| Glu Val Gln Leu Gln Gln Ser Gly Pro | (SEQ ID NO: 3) |
| 1 5 | |
| Glu Leu Val Lys Pro Gly Ala Ser Val | |
| 10 15 | |
| Lys Met Ser Cys Lys Ala Ser Gly Tyr | |
| 20 25 | |
| Thr Phe Thr Ser Tyr Val Met His Trp | |
| 30 35 | |
| Val Lys Gln Lys Pro Gly Gln Gly Leu | |
| 40 45 | |
| Asp Trp Ile Gly Tyr Ile Val Pro Tyr | |
| 50 | |
| Asn Asp Gly Thr Lys Tyr Asn Glu Lys | |
| 55 60 | |
| Phe Lys Gly Lys Ala Thr Leu Thr Ser | |
| 65 70 | |
| Asp Lys Ser Ser Ser Thr Ala Tyr Met | |
| 75 80 | |
| Glu Leu Ser Arg Leu Thr Ser Glu Asp | |
| 85 90 | |
| Ser Ala Val Tyr Tyr Cys Val Tyr Gly | |
| 95 | |
| Ser Arg Tyr Asp Trp Tyr Leu Asp Val | |
| 100 105 | |
| Trp Gly Ala Gly Thr Thr Val Thr Val | |
| 110 115 | |
| Ser Ser | |
| mSM5-1 light chain variable | |
| region amino acid sequence | |
| NIMMTQSPSSLAVSAGEKVTMSCKSSQSVLYSSNQK | (SEQ ID NO: 4) |
| NYLAWYQQKPGQSPKLLIYWASTRESGVPDRFTGSG | |
| SGTDFTLTISSVQAEDLAVYYCHQYFSSYTFGGGTK | |
| LEIKR | |
| mSM5-1 heavy chain variable | |
| region nucleotide sequence | |
| GAGGTCCAGCTGCAGCAGTCTGGACCTGAGCTGGTA | (SEQ ID NO: 7) |
| AAGCCTGGGGCTTCAGTGAAGATGTCCTGCAAGGCT | |
| TCTGGATACACATTCACTAGCTATGTTATGCACTGG | |
| GTGAAGCAGAAGCCTGGGCAGGGCCTTGACTGGATT | |
| GGATATATTGTTCCTTACAATGATGGCACTAAGTAC | |
| AATGAGAAGTTCAAAGGCAAGGCCACACTGACTTCA | |
| GACAAATCCTCCAGCACAGCCTACATGGAGCTCAGC | |
| AGACTGACCTCTGAGGACTCTGCGGTCTATTATTGT | |
| GTCTACGGTAGTAGGTACGACTGGTATTTAGATGTC | |
| TGGGGCGCAGGGACCACGGTCACCGTCTCCTCA | |
| mSM5-1 light chain variable | |
| region nucleotide sequence | |
| AACATTATGATGACACAGTCGCCATCATCTCTGGCT | (SEQ ID NO: 8) |
| GTGTCTGCAGGAGAAAAGGTCACTATGAGCTGTAAG | |
| TCCAGTCAAAGTGTTTTATACAGTTCAAATCAGAAG | |
| AACTACTTGGCCTGGTACCAGCAGAAACCAGGGCAG | |
| TCTCCTAAACTGCTGATCTACTGGGCATCCACTAGG | |
| GAATCTGGTGTCCCTGATCGCTTCACAGGCAGTGGA | |
| TCTGGGACAGATTTTACTCTTACCATCAGCAGTGTA | |
| CAAGCTGAAGACCTGGCAGTTTATTACTGTCATCAA | |
| TATTTCTCCTCATACACGTTCGGAGGGGGGACCAAG | |
| CTGGAAATAAAGCGG | |

2. Construction and Expression of Chimeric Antibody

The variable regions of the heavy and the light chain of mSM5-1 shown above were used to construct mouse-human chimeric antibody. The chimeric antibody expression vector was constructed in an identical manner to huSM5-1 described in Example 3.

Prior to transfection, CHOdhfr-cells were maintained in complete DMEM medium containing glycin, hypoxanthine and thymidine (GHT). The expression vector pMG18-3K containing heavy and light chain of chSM5-1 was transfected into CHOdhfr-cells using Lipofectamine 2000 reagent (Invitrogen, Garlsbad, Calif.) according to the manufacture's instruction. The transfected cells were then selected in GHT free DMEM medium containing stepwise increments in MTX level up to 1.0 M. Drug resistant clones were picked and expanded for further analysis. The culture supernatants from cell clones were analyzed for antibody production by the sandwich ELISA which used goat anti-human IgG (Fc) (KPL) as capture antibody and goat anti-human kappa-HRP (KPL) as detector antibody. Purified human IgG1/Kappa (Sigma) was used as a standard in the ELISA assay. The clone producing the highest amount of antibody was selected and grown in serum-free medium. The recombinant antibodies were purified by Protein A affinity chromatography from the serum-free culture supernatant.

3. Construction and Expression of Humanized Antibody

The V_{H} of human antibody KOL was chosen as framework for the humanized heavy chain and the V_{L} of human Bence-Jones protein REI was chosen for the humanized light chain. The three CDRs from mSM5-1 light chain or heavy chain were directly grafted into human antibody light chain or heavy chain framework regions to generate a humanized antibody genes. The light and heavy variable region genes of humanized antibodies were synthesized by overlapping PCR method. The expression vectors for humanized antibodies were constructed in an identical manner to the chimeric antibody described above.

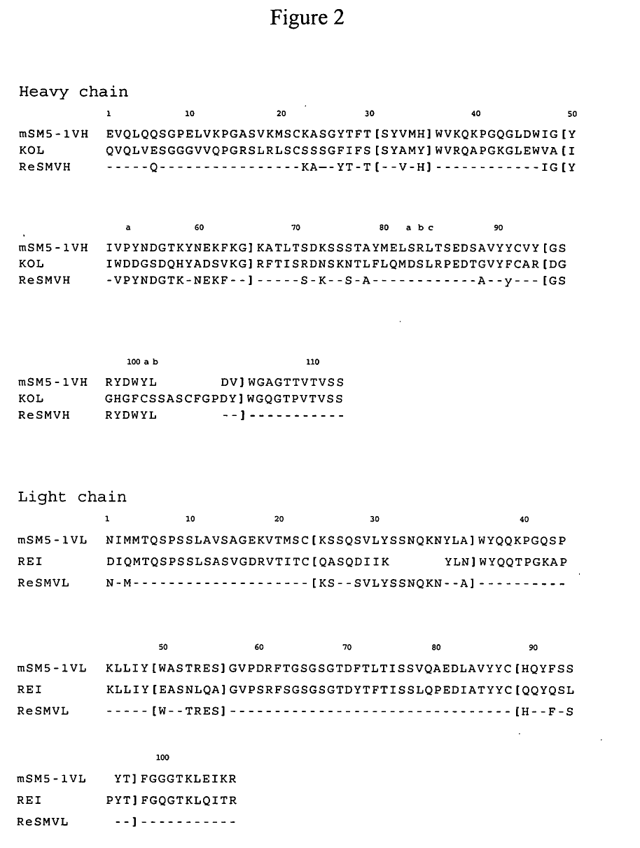

As shown in FIG. 2, the three CDRs from mSM5-1 light chain or heavy chain were directly grafted into human antibody light chain or heavy chain framework regions to generate humanized antibody genes. The humanized V_{L} and V_{H} were cloned into pMG18-3K expression vector and was expressed transiently in COS cells, yielding humanized version. Humanized antibody in COS cell culture supernatant was quantitated by ELISA and the binding of this version to hepatocelluer carcinoma cell line QYC was determined by FCM. The antigen binding activity assay indicated that this antibody bound poorly to human melanoma cells. This suggested that some human FR residues must be altered to reconstitute the full binding activity. The important FR residues that may have influences on binding activity were analyzed and the backmutation assay was carried out. Finally a humanized antibody showing the same antigen binding activity as chSM5-1 was obtained. The humanized version was designated as ReSM5-1 and its amino acid sequence and nucleotide sequence of both the heavy chain and the light chain shown in Table 3 below. In the competition binding assay, ReSM5-1 displayed equivalent avidity as the murine SM5-1 or chimeric SM5-1 antibody.

TABLE 3

Amino acid and nucleotide sequences for humanized
anti-SM5-1 antibody (ReSM5-1) variable regions

| | |
|---|---|
| ReSM5-1 heavy chain variable | |
| region amino acid sequence | |
| Gln Val Gln Leu Val Gln Ser Gly Gly | (SEQ ID NO: 1) |
| 1 5 | |
| Gly Val Val Gln Pro Gly Arg Ser Leu | |
| 10 15 | |
| Arg Leu Ser Cys Lys Ala Ser Gly Tyr | |
| 20 25 | |
| Thr Phe Thr Ser Tyr Val Met His Trp | |
| 30 35 | |
| Val Arg Gln Ala Pro Gly Lys Gly Leu | |
| 40 45 | |
| Glu Trp Ile Gly Tyr Ile Val Pro Tyr | |
| 50 | |
| Asn Asp Gly Thr Lys Tyr Asn Glu Lys | |
| 55 60 | |
| Phe Lys Gly Arg Phe Thr Ile Ser Ser | |
| 65 70 | |
| Asp Lys Ser Lys Ser Thr Ala Phe Leu | |
| 75 80 | |
| Gln Met Asp Ser Leu Arg Pro Glu Asp | |
| 85 90 | |
| Thr Ala Val Tyr Tyr Cys Ala Arg Gly | |
| 95 | |
| Ser Arg Tyr Asp Trp Tyr Leu Asp Tyr | |
| 100 105 | |
| Trp Gly Gln Gly Thr Pro Val Thr Val | |
| 110 115 | |
| Ser Ser | |
| ReSM5-1 light chain variable | |
| region amino acid sequence | |
| Asn Ile Met Met Thr Gln Ser Pro Ser | (SEQ ID NO: 2) |
| 1 5 | |
| Ser Leu Ser Ala Ser Val Gly Asp Arg | |
| 10 15 | |
| Val Thr Ile Thr Cys Lys Ser Ser Gln | |
| 20 25 | |
| Ser Val Leu Tyr Ser Ser Asn Gln Lys | |
| 30 35 | |
| Asn Tyr Leu Ala Trp Tyr Gln Gln Thr | |
| 40 45 | |
| Pro Gly Lys Ala Pro Lys Leu Leu Ile | |
| 50 | |
| Tyr Trp Ala Ser Thr Arg Glu Ser Gly | |
| 55 60 | |
| Val Pro Ser Arg Phe Ser Gly Ser Gly | |
| 65 70 | |
| Ser Gly Thr Asp Tyr Thr Phe Thr Ile | |
| 75 80 | |
| Ser Ser Leu Gln Pro Glu Asp Ile Ala | |
| 85 90 | |
| Thr Tyr Tyr Cys His Gln Tyr Phe Ser | |
| 95 | |
| Ser Tyr Thr Phe Gly Gln Gly Thr Lys | |
| 100 105 | |
| Leu Gln Ile Thr Arg | |
| 110 | |
| ReSM5-1 heavy chain variable | |
| region nucleotide sequence | |
| CAGGTGCAGCTGGTGCAGTCTGGCGGTGGAGTGGTC | (SEQ ID NO: 5) |
| CAGCCCGGCCGCAGCCTGAGGCTGTCCTGCAAGGCA | |
| TCTGGCTACACCTTCACCAGCTACGTGATGACATGG | |
| GTGCGCCAAGCCCCCGGAAAGGGCCTCGAATGGATT | |
| GGCTACATTGTGCCTTATAATGACGGTACTAAGTAC | |
| AATGAAAAGTTCAAGGGCAGATTTACAATATCAAGT | |
| GACAAGAGCAAGTCAACCGCATTCCTCCAAATGGAC | |
| AGCTTGCGTCCAGAGGACACCGCCGTATACTATTGT | |
| GTGCGCGGCAGCCGTTACGACTGGTACTTGGACTAC | |
| TGGGGCCAAGGCACTCCAGTCACCGTCTCCTCT | |
| ReSM5-1 light chain variable | |
| region nucleotide sequence | |
| AACATCATGATGACTCAGAGCCCATCCAGCTTGAGC | (SEQ ID NO: 6) |
| GCATCAGTAGGCGACCGCGTAACGATCACTTGCAAA | |
| TCCTCTCAGTCAGTATTGTACTCCAGCAACCAGAAG | |
| AACTACCTGGCCGGATATCAGCAGACTCCCGGCAAA | |
| GCCCCAAAGTTGCTGATTTATTGGGCCTCCACGCGC | |
| GAGTCTGGCGTGCCATCACGCTTTAGCGGCAGCGGG | |
| TCCGGTACAGATTACACGTTTACCATTAGCAGTCTG | |
| CAGCCTGAGGACATAGCCACCTACTACTGTCACCAG | |
| TACTTTAGTTCCTACACTTTTGGCCAGGGAACTAAA | |
| CTGCAGATTACTCGA | |

4. Purification of Humanized Antibodies.

Prior to transfection, CHOdhfr-cells were maintained in complete DMEM medium containing glycin, hypoxanthine and thymidine (GHT). Appropriate expression vector was transfected into CHOdhfr-cells using Lipofectamine 2000 reagent (Invitrogen, Garlsbad, Calif.) according to the manufacture's instruction. The transfected cells were then selected in GHT free DMEM medium containing stepwise increments in MTX level up to 1.0 M. Drug resistant clones were picked and expanded for further analysis. The culture supernatants from cell clones were analyzed for antibody production by the sandwich ELISA which used goat anti-human IgG (Fc) (KPL) as capture antibody and goat anti-human kappa-HRP (KPL) as detector antibody. Purified human IgG1/Kappa (Sigma) was used as a standard in the ELISA assay. The clone producing the highest amount of antibody was selected and grown in serum-free medium. The recombinant antibody (ReSM5-1) was purified by Protein A affinity chromatography from the serum-free culture supernatant.

### Example 5

### Biological Activity of Monoclonal Antibody huSM5-1

1. Effect of huSM5-1 on Melanoma Cells

The melanoma cell line A2058 was expanded to 10⁶/ml in RPMI-1640 culture medium. The cell suspension (20 μl) and different amount of huSM5-1 (0, 1, 5, 10, 20, 50, 100 μl) were added into each well of the 96 well plate. The antibody huSM5-1 was diluted in RPMI-1640 medium contianing 10% FCS to reach a final concentration of 1 μg/μl before it was added into the well. RPMI-1640 medium contianing 10% FCS was added to each well to make a final volume of 500 μl in each well. Each condition was performed in triplicate. Cells were incubated at 37° C. in a humidified incubator at 5% CO₂ for 24 h. At the end of the incubation, cells were suspended and viable cells were examined under microscope or assessed using MTT.

(1) Viable Cell Count.

Viable cells in 0.2 ml cell suspension from each well were counted. The results are shown in Table 4. Viable cells are expressed as percentage of viable cells compared to the wells without addition of huSM5-1. As shown in Table 4, antibody huSM5-1 significantly reduced number of viable A2058 cells in a dose dependent manner after 24 h incubation.

TABLE 4
Viable cells after treatment with antibody huSM5-1

| huSM5-1 | | | | | | | |
|---|---|---|---|---|---|---|---|
| addition (μl) | 0 | 1 | 5 | 10 | 20 | 50 | 100 |
| Cell survival | 100 | 87.8 | 68.8 | 53.2 | 42.6 | 32.6 | 22.6 |
| (%) | | | | | | | |

MTT solution (5 mg/ml) was added into 0.2 ml cell suspension describe above at 1:10 dilution. The cells were then incubated at 37° C. for 30 min and optical adsorption at 570 nm for each sample was read. As shown in Table 5, antibody huSM5-1 treated cells had significant lower optical absorption at 570 nm compared to control, indicating viable cells were significantly reduced after treatment with antibody huSM5-1.

TABLE 5
Percentage of viable cells after huSM5-1 treatment assessed by MTT assay

| huSM5-1 | | | | | | | |
|---|---|---|---|---|---|---|---|
| addition(ul) | 0 | 1 | 5 | 10 | 20 | 50 | 100 |
| Optical | 100.0 | 96.3 | 84.9 | 66.2 | 47.7 | 33.6 | 20.1 |
| adsorption | | | | | | | |
| (%) | | | | | | | |

The above results showed that huSM5-1 antibody can prevent proliferation of human melanoma cells effectively in vitro and may be used for treating melanoma.

2. Flow Cytometry Analysis of huSM5-1 Antigen Expression

Expression of human SM5-1 antigen on cancer cells' surface were conducted using flow cytometry with huSM5-1 antibody. Cancer cells used in the study included breast cancer cell lines SK-BR-3, MDA-MB-231, BT-20-T, MDA-MB-468 and MCF-7; melanoma cell lines CRL-1872 and U10; hepatocellular carcinoma cell lines QYC, LYX and XJC.

In addition to the above researches, we also conducted the following studies with the acquired cell line and found that SM5-1 antigen is not only expressed characteristically by melanoma cells but also over-expressed by breast cancer and hepatocellular carcinoma cells.

Cancer cells to be tested were incubated with appropriately diluted huSM5-1 for 1 h, and then washed 5 times with PBS. Cells were incubated with FITC labeled goat-anti-human IgG (2 mg/ml, Jackson Immunoresearch Laboratories, West Grove, Pa.) and then fixed in PBS containing 1% formalin. Expression of the antigen in these cells were then analyzed using flow cytometry.

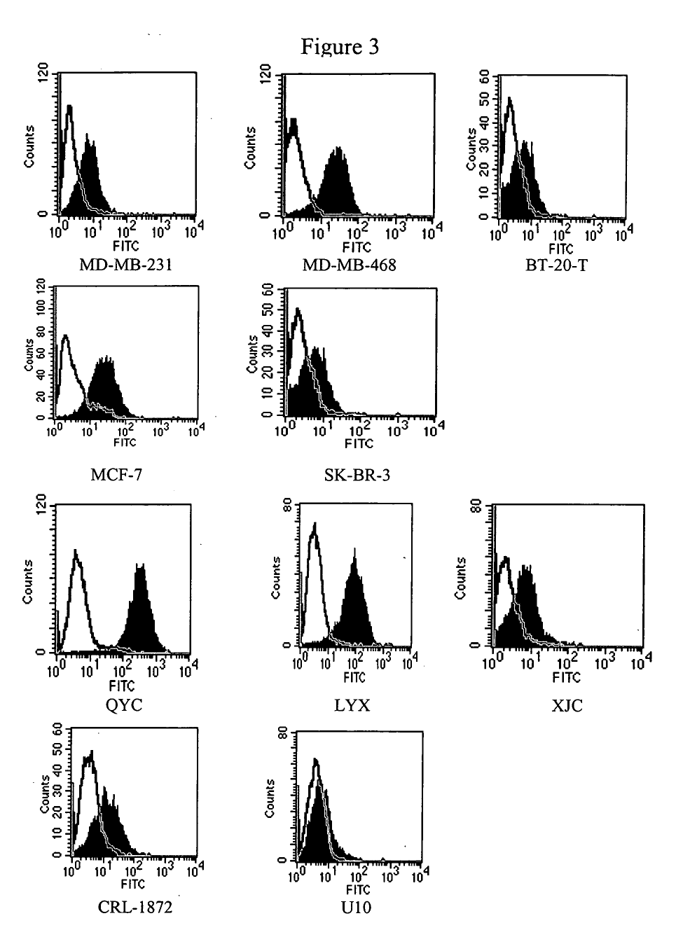

As shown in FIG. 3, SM5-1 antigen was highly or moderately expressed in breast cancer cell lines (MDA-MB-231, MDA-MB-468, and MCF-7), melanoma cell lines (CRL-1872), and hepatocellular carcinoma cell lines (QYC and LYX). But the antigen had a lower level expression in hepatocellular carcinoma cell line XJC, breast cancer cell lines SK-BR-3, BT-20-T, melanoma cell line U10. These data indicated that the human SM5-1 antigen was expressed in melanoma, breast cancer, and hepatocellular carcinoma cell lines.

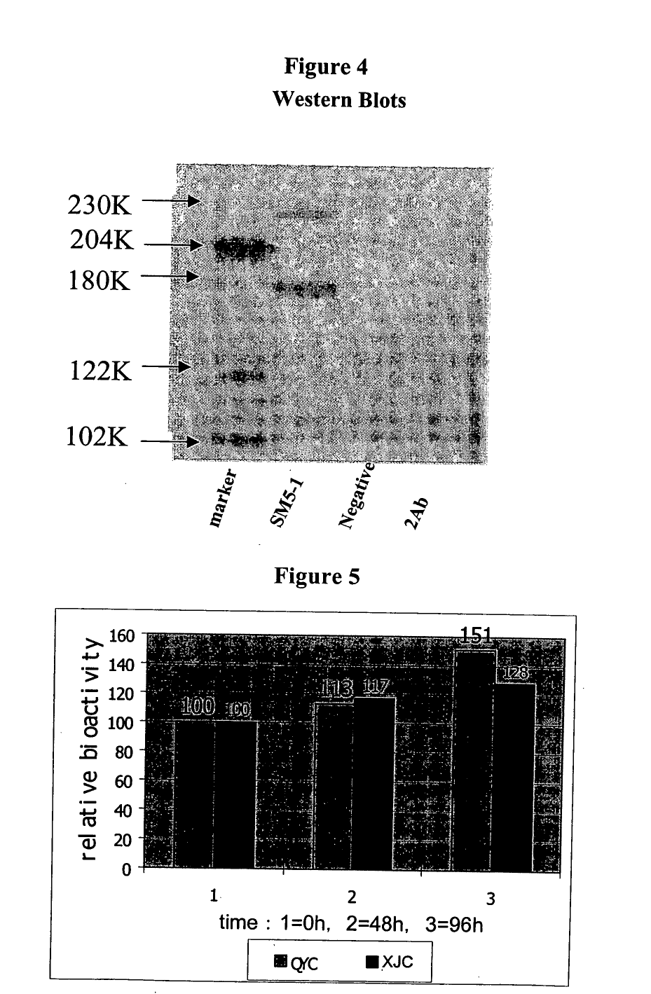

To identify the antigenic determinants for huSM5-1 antibody, proteins from hepatocellular carcinoma cell line QYC were extracted and immunoprecipitated with huSM5-1 antibody. Mouse anti-human CD4 antibody was also used for immunoprecipitation as a negative control. Immunoprecipitates were analyzed by western blotting. The blot was probed with huSM5-1 antibody. As shown in FIG. 4, two proteins with molecular weight of 230 kD and 180 kD were found only when the proteins were immunoprecipitated with huSM5-1 antibody; no specific bands were observed in the position of negative control antibody and secondary antibody.

3. huSM5-1 Antibody Induces Caspase 10-Related Apoptosis

To determine whether caspase is involved in huSM5-1 antibody induced apoptosis, a variety of caspase inhibitors were tested to observe the inhibitory rate of apoptosis. The caspase inhibitors tested included common caspase inhibitor (Z-VAD-FMK), caspase-1 inhibitor (Z-WEHD-FMK), caspase-2 inhibitor (Z-VDVAD-FMK), caspase-3 inhibitor (Z-DEVD-FMK), caspase-4 inhibitor (Z-YVAD-FMK), caspase-6 inhibitor (Z-VEID-FMK), caspase-8 inhibitor (Z-IETD-FMK), caspase-9 inhibitor (Z-LEHD-FMK), caspase-10 inhibitor (Z-AVED-FMK), caspase-13 inhibitor (Z-LEED-FMK). QYC cells were incubated with caspase inhibitor at 50 mol/l for 2 h. QYC cells were then treated with 50 ng/ml huSM5-1 antibody. The inhibitory rates of these inhibitors were: 72% for pan-caspase inhibitor, 52% for caspase-10 inhibitor, 28% for caspase-6 inhibitor, 27% for caspase-1 inhibitor, 17% for caspase-8 inhibitor, 15% for caspase-13 inhibitor, 14% for caspase-4 inhibitor, 5% for caspase-9 inhibitor, 1% for caspase-2 inhibitor, 1% for caspase-3 inhibitor. Based on the above result, pan caspase inhibitor had the highest inhibitory effect on huSM5-1 induced apoptosis, and caspase 10 inhibitor also significantly inhibited the huSM5-1 induced apoptosis, indicating that huSM5-1 induced apoptosis was related to caspase-mediated pathway, and caspase-10 was one of the caspases which affected huSM5-1 induced apoptosis mostly.

To further confirm that huSM5-1 induced apoptosis is caspase-10 related, caspase-10 color comparing analysis kit was used to examine the increase of caspase-10 bioactivity in QYC and XJC cells. Caspase-10 activity was examined with caspase-10 analysis kit (R&D, USA) according to manufacture's instruction. Cells were incubated with 50 ng/ml huSM5-1 antibody for a certain period of time. Cells were then centrifuged and lysed in a lysis buffer (25 ul/10⁶ cells) on ice for 10 min. The lysates were centrifuged, and the supernatant was transferred into new tubes and kept on ice. The enzyme activity of caspase was examined on 96-well micro-titer plate. Each reaction included 501 μl supernatant from cell lysate, 50 μl 2× reaction buffer, 10 μl fresh DTT storage solution. In addition, 5 ul caspase-10 color-comparing substrate (AEVD-pNA) was added into the reaction. The plate was incubated at 37° C. for 1-2 h and absorbance at 405 nm was then measured. The increase of caspase-10 related activity was calculated according to the following equation:
Caspase-10 activity (%)=(B−C)/(A−C)×100%

“A” represents OD value of supernatant from cell lysates without huSM5-1 antibody treatment; “B” represents OD value of supernatant from cell lysate treated with huSM5-1; “C” represents OD value of negative control. Each test was done in triplicate.

FIG. 5 shows that the caspase-10 activity increased from 13% (48 h) to 51% (96 h) for huSM5-1 treated QYC cells and from 17% (48 h) to 38% (72 h) for huSM5-1 treated XJC cells. However, the caspase-10 activity decreased to 28% for XJC cells at 96 h. These data indicated that huSM5-1 induced apoptosis is caspase-10 related.

4. Effect on Cell Differentiation and Growth of huSM5-1

Cell growth inhibition by huSM5-1 antibody was tested using MTT assay. MTT assay is based on the principle that viable cells can reduce yellow MTT into blue purple crystals. Cells of interest (1×10³) were incubated with various concentrations of antibodies. After a period of time, 20 μl MTT (0.5 mg/ml) was added into the cell culture medium and was incubated for 2 h. After the incubation, culture medium was removed and 150 μl DMSO was added to solubilize the MTT precipitate. The reduced MTT was examined by measuring absorbance at 490 nm using Benchmark optical absorption reading machine (Bio-Rad Laboratories). The cell growth inhibition was calculated according to the following equation:
Inhibition (%)=(B−C)/(A−C)×100

“A” represents the OD value of cells without huSM5-1 treatment; “B” represents the OD value of huSM5-1 treated cells; and “C” represents the OD value of negative control. Each condition was performed in triplicate.

Growth inhibition of huSM5-1 were tested in four tumor cell lines (hepatocellular carcinoma cell QYC and XJC, breast cancer cell line MDA-MB-231, and melanoma cell line CRL-1872) using MTT assay. Cells were treated with different amount of huSM5-1 (50 ng/ml, 10 ng/ml, 1 ng/ml) for 24 h, 48 h, 72 h, and 96 h. The most inhibition occurred when the concentration of the antibody was at 50 ng/ml and after 72 h treatment. For example, at 50 ng/ml, 10 ng/ml and 1 ng/ml, growth inhibition of hepatocellular carcinoma cell line QYC was 29%, 11% and 7% respectively, after 24 h treatment with huSM5-1; after 48 h treatment, growth inhibition was about 28%, 17% and 5% respectively; after 72 h, inhibition was 43%, 19% and 10%; and after 96 h, inhibition was 36%, 11% and 2.5%. Similar results were observed with other three cell lines. The antibody used for negative control was an unrelated human Ig G1, which showed no effect on cell growth. The above results showed that huSM5-1 antibody could significantly inhibited the growth of tumor cells in a dose- and time-dependent manner.

As shown above, the growth inhibition of huSM5-1 antibody is related to apoptosis induction through a caspase-10 mediated pathway, and the apoptotic process may involve DNA fragmentation.

### Example 6

### The In vitro Effect on Tumor Cells of Anti-Human SM5-1 Chimeric Antibody (chSM5-1) and Humanized Antibody (ReSM5-1)

The cell lines used in viability examination was QYC cells which were obtained from Shanghai International Joint Cancer Institute. The QYC cells was cultured in 25 cm² flask with RPMI-1640/DMEM(V:V=1:1, GIBCO) containing 10% FBS (GIBCO).

The above cells were digested with 0.05% trypsin and in 0.02% EDTA, and cell number was counted and adjusted to 6×10⁴/ml in RPMI-1640/DMEM culture medium containing 10% FCS.

The assay was performed in 96-well plate. The anti-human SM5-1 humanized and chimeric antibody described above were diluted with RPMI-1640/DMEM containing 10% FCS. The antibody to be tested (20 mg/ml) was diluted to to 8 μg/ml in serial dilution, with each dilution no more than 10-fold. The antibody were further diluted 1:2 serially with RPMI-1640/DMEM containing 10% FCS for fourteen serial concentrations in 96-well plate, leaving 100 μl/well. Cells (100 μl) were added into each well containing 200 μl various concentration of antibody or 200 μl of RPMI-1640/DMEM containing 10% FCS as control. In order to prevent edge effect of the plate, wells that were at the edge of the plate were not used for the assay but added 200 μl PBS. The plate was incubated at 37° C. with 7% CO₂ for seven days.

Color developing reagent PMS:MTS (1:20) (20 μl) was added into each well of the 96-well plate. The plate without the lid was then incubated for 3 h.

The 96-well plate was read at OD490 nm. The results were expressed in the following 4-parameter equation:
Y=(A−B)/[1+(X/C)^{D}]+B

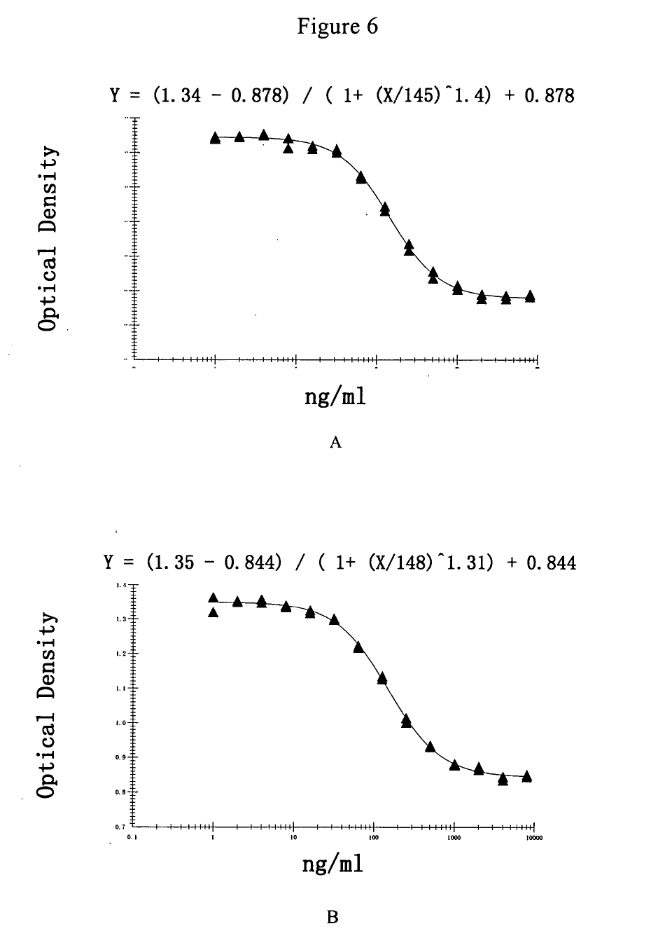

According to the equation, when X=∞, Y=B, the upper limit; when X=0, Y=A, the lower limit; when X═C, Y=(A+B)/2, the half of the maximum. Therefore, C is the half effective dose (ED50). FIG. 6 shows that the growth inhibition of tumor cell line (QYC) by humanized as well as chimeric anti-human SM5-1 antibody.

The test showed that chimeric and humanized anti-SM5-1 antibodies significantly inhibited in vitro proliferation of QYC cells.

### Example 7

### Antibody-Dependent Cell Mediated Cytotoxicity (ADCC) by ReSM5-1/Chimeric SM5-1

1. Isolation of Peripheral Blood Lymphocytes (PBL)

Venous blood was taken sterilely from healthy donor and put into sterile 15 ml centrifuge tube containing 20 U/ml heparin. The solution was carefully mixed and equal volume of steriled PBS was added to dilute the blood.

In a 15 ml centrifuge tube, 6 ml room temperature pre-warmed 100% lymphocytes isolation fluid (obtained from CACS, Cellular Biology Institute) was added. Along the tube wall of the tilted tube, 6 ml diluted anti-coagulation peripheral blood was added slowly into the tube without damaging the interface.

The tube was centrifuged at 20° C., 800 g for 30 min with brake turned off. After the centrifuge, three layers were formed. The three layers (from above to the bottom) were blood plasma layer, cell separation liquid layer, red and granular cell layer. A white frosted glass-like layer between the plasma layer and cell separation liquid layer was the layer containing lymphocytes and mononuclear cells. This white layer was taken out using a pipette and put into another 15 ml sterile centrifuge tube. PBS was added into the centrifuge tube to dilute the PBL suspension and then centrifuged at 200 g for 5 min. The pellet was washed 2 times with PBS.

The cell concentration was adjusted with non-phenol red RPMI-1640/DMEM (GIBCO) to 6×10⁶/ml. Suspended cells in 15 ml centrifuge tube were incubated at 37° C. with 7% CO₂ for further study.

2. Preparation for Target QYC Cells

QYC cells at the logistically growing stage were taken out from incubator. Cells were washed 2 times with PBS. 0.5 ml of digestion fluid containing 0.05% trypsin and 0.02% EDTA was added into the cells. Morphology of the cells were observed under microscope. When cells began to become round, digestion fluid was removed. Cells were resuspended in non-phenol red RPMI-1640/DMEM. Cells were counted, and the concentration was adjusted to 3×10⁵/ml.

3. The Role of SM5-1 Antibodies on Cells

The anti-SM5-1 antibodies was diluted to 40 μg/ml, and was then diluted serially 1:2 in 1.5 ml centrifuge tube with non-phenol red RPMI-1640/DMEM, totaling 14 concentrations and 300 ul/tube. QYC cells (300 μl) were added into each tube. Cells were incubated at 4° C. for 30 min. Cells were then centrifuged at 200 g for 5 min. Cell pellets were washed 2 times with PBS. Cells were then suspended in 300 ul non-phenol red RPMI-1640/DMEM. Cells reacted with anti-SM5-1 antibody were added into a well of a 96-well plate at 100 ul/well. Effector cells at 100 ul/well were added into each well of the 96-well plate with effector to target ratio of 20:1. The plate was incubated at 37° C. with 7% CO₂ for 7 h.

4. Color Developing and OD₄₉₀

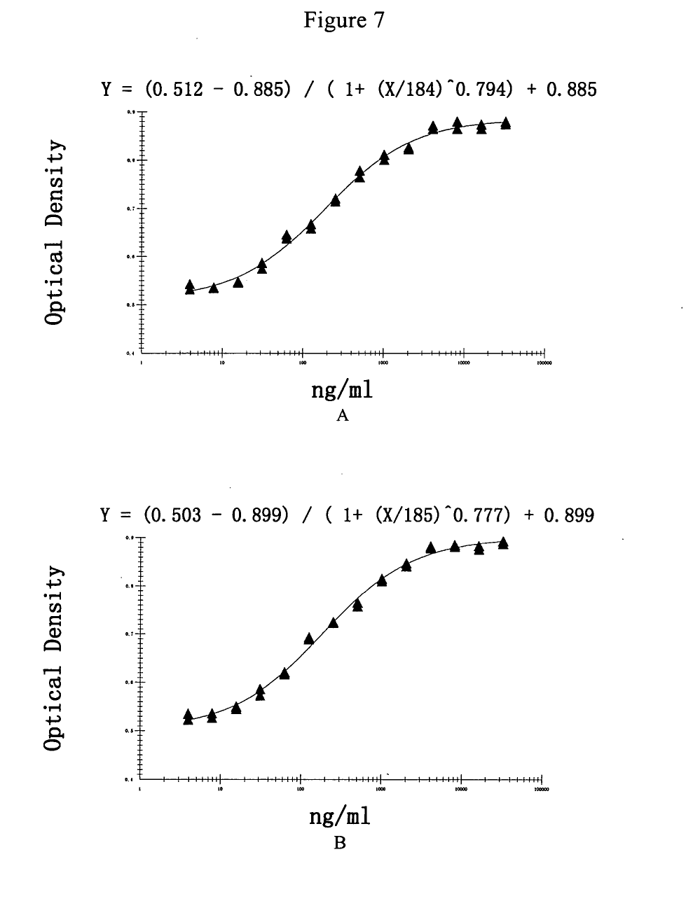

Cytotoxicity Detection Kit by Roche's Corporation was used. The catalyst was dissolved in 1 ml ddH₂O. The catalyst was mixed with dye solution at a ratio of 1:45. The 96-well plate taken from the incubator was centrifuged at 200 g for 5 min, and then 50 ul supernatant was taken from each well and added into another 96-well plate. Mixed color-developing fluid 50 ul/well was added into 96-well plate, and the plate was incubated at room temperature (avoiding light) for 30 min. OD was read at 490 nm. Result in FIG. 7 indicated that chimeric and humanized SM5-1 mAbs induced the apoptosis and inhibit the growth of tumor cells through ADCC pathway.

### Example 8

### Complement-Dependent Cytotoxicity (CDC)

Antigen recognized by anti-SM5-1 is highly expressed on the surface of human hepatocellular carcinoma cell line QYC. With human complement in the culture fluid, the target cells bound with chimeric antibodies will be lysed by so-called complement dependent cell mediated cytotoxicity (CDC). When there is an over dose of complement (provided by fresh normal human sera), in a certain range, the degree of lysis is related to antibody concentration. Degree of lysis can be determined by detecting lactate dehydrogenase (LDH) released by the lysed cells.

Cell line for bioactivity determination is QYC, which is without any pathogen. The cells were cultured in 25-75 cm² flasks with RPMI-1640/DMEM (1:1) containing 10% NBS. The following culture medium were prepared and stored at 4° C.: A, RPMI-1640/DMEM (1:1) containing 10% NBS; B, non-phenol red RPMI-1640 without sera; C, culture fluid B with 5% normal human sera. The sera were freshly isolated from healthy donor, and was stored at −80° C. The culture condition was 37° C., 5% CO₂, and saturation humidity.

QYC cells in the logistic growth stage were taken and counted. For bioactivity determination, 2×10⁶ cells were used for each 96-well plate. Cells were centrifuged and the supernatant was removed. Culture medium B was added to the pellet to resuspend the cells and the cell concentration was adjusted to 2×10⁵/ml. Resuspended cells were added into 96-well plate at 0.1 ml/well. Wells near the edges were not used and sterile water was added into these wells to avoid the edge effects.

A standard sample and the protein to be tested were diluted to 40 μg/ml in serial dilution, with each dilution no more than 10-fold. The standard and the protein to be tested were further diluted 1:2 in fourteen 1.5-ml sterile centrifuge tubes with the final dilution volume 0.4 ml. (Note: the highest concentration added into a 96-well plate was 2 μg/ml).

Culture medium C was used as negative control. In the culture plate that QYC cells were seeded, 0.1 ml of the diluted standard protein or the protein to be tested described above or the negative control was added into each well. Each condition was done in duplicate. The plate was incubated at 37° C., 5% CO₂ for 3-4 h.

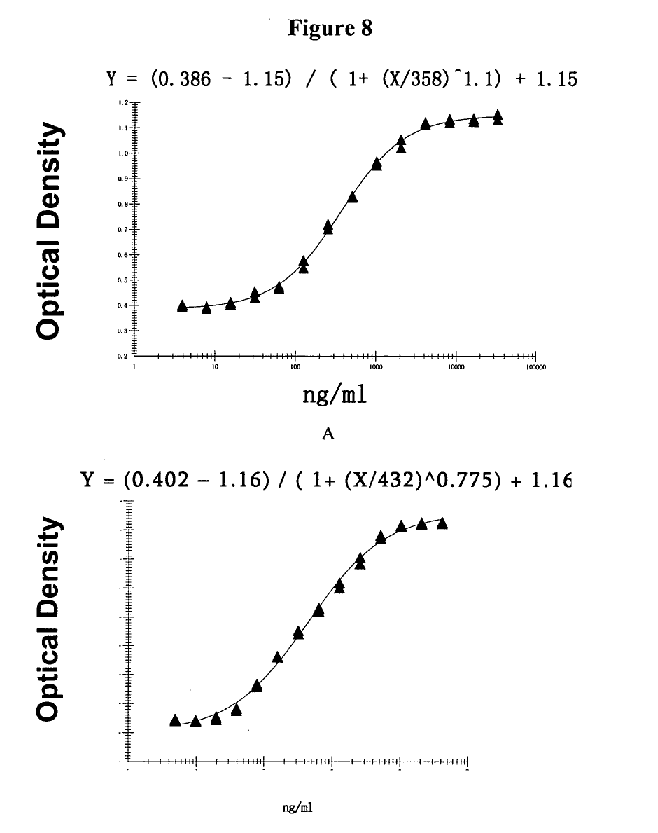

50 ul supernatant was transferred from each well to corresponding wells in a second 96-well plate, and 50 ul well-mixed LDH test kit reagent was added into the well of the second plate. The second plate was incubated at room temperature for 0.5 h without light. The color developing was stopped by adding 50 μl neutralizing fluid (acetic acid 1 mol/L). OD was measured with 490 nm as detecting light wave length, and 630 nm as reference wave length. FIG. 8 shows the result.

The results were analyzed with specific analysis software Select2.2 to make auto analysis and calculate standard curve line: horizontal axis stands for the concentration of the standard product and samples, vertical axis stands for optical adsorption, recovery equation as 4-parameter equation, resulting in a “s” curve line. Half effective dosage (ED50) of the standard product and samples were calculated. The bioactivity of the samples was shown as the following.

Bioactivity percentage (%)=half effective dosage of standard product (ED50)/HALF effective dosage of samples (ED50)×100%

Note: the software gave the following 4-parameter equation:
Y=(A−B)/[1+(X/C)^{D}]+B

According to the equation, when X=+∞, Y=B, the upper limit; when X=0, Y=A, the lower limit; while X=C, Y=(A+B)/2, the half of the maximum. Therefore, C is the half effective dose (ED50).

As shown in FIG. 8, both chimeric and humanized anti-SM5-1 antibody induced the apoptosis and inhibited the growth of tumor cells through the CDC pathway.

### Example 9

### The Therapeutic Effect of Anti-SM5-1 Monoclonal Antibodies for QYC-Bearing Nude Mice

Chimeric and humanized anti-SM5-1 monoclonal antibodies, and humanized and chimeric anti-CD3 antibodies were tested for their therapeutic effects in QYC bearing nude mice.

Forty female nude mice were inoculated s.c with QYC. After seven weeks, tumor masses reached 0.5 cm in diameter. These mice were randomly divided into 5 groups: 8 mice for PBS group; 8 mice for non-related antibody, chimeric anti-human CD3 mAb, 4 mg/kg; 8 mice for humanized anti-human CD3 mAb, 4 mg/kg; 8 mice for anti-human SM5-1 chimeric mAb, 4 mg/kg; 8 mice for anti-human SM5-1 humanized mAb, 4 mg/kg.

Four mAbs were diluted into final concentration 0.4 mg/ml with PBS. Mice were tail vein injected at 4 mg/kg/week through tail vein, with the control injected equal volume of PBS. According to body weight of nude mice, injection volume was about 250 ul for each nude mouse.

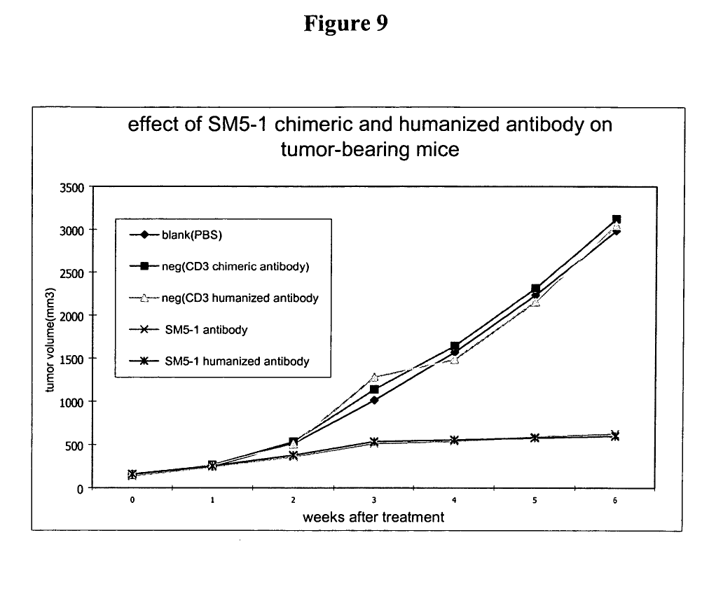

After 6 weeks, size of the tumor mass was measured in each mouse and statistical analyzed. The results are shown in FIG. 9. FIG. 9 indicated that both chimeric and humanized anti-human SM5-1 monoclonal antibodies were effective in controlling the size of the tumor mass formed by human hepatocellular carcinoma cell line QYC. These antibodies may function via ADCC and/or CDC.

### Example 10

### Tissue Distribution of ¹²⁵I, Labeled Anti-Human SM5-1 Chimeric and Humanized mAbs after Tail Vein Injection into Nude Mice

Eight nude mice bearing tumor averaging 0.7 cm in diameter were divided randomly into two groups. According to literatures and clinical dosage, 4 mg/kg was enough to inhibit the growth of tumors in nude mice. Thus, single dosage was used to study the in vivo distribution.

The labeling efficiency was 682823 cpm/μl (0.52 μg/μl) for anti-human SM5-1 chimeric antibody, 681012 cpm/μl (0.52 μg/μl) for anti-human SM5-1 humanized mAbs.

The nude mice were weighed and ¹²⁵I labeled humanized and chimeric anti-human SM5-1 mAbs were injected through tail vein at about 180 ul for each mouse. According to literature, distribution measurement was generally done within 24-72 h. Forty-eight hours was taken as the measure time in this experiment.

Firstly, the blood was taken from eyeballs with eye forceps, and then the following 21 kinds of tissues (blood, thyroid gland, lung, heart, skin, gallbladder, spleen, fat, adrenal gland, kidney, liver, stomach, intestine, intestinal content, mesentery lymphnode, bladder, testis, muscle, bone, brain, and tumor) were taken sequentially. Tumor tissue was taken at last. Cross pollution should be avoided during the process.

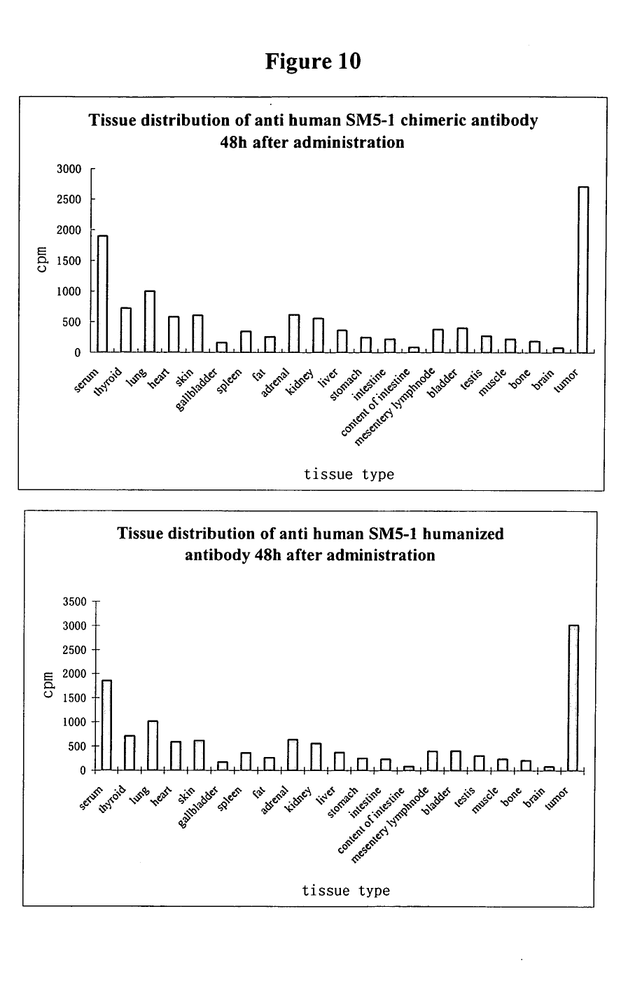

Each tissue was placed in the tubes and weighed. The cpm was read using γ-counter. For each tissue, mg/cpm was calculated. FIG. 10 shows the results. As shown in FIG. 10, both chimeric and humanized anti-human SM5-1 mAbs were selectively concentrated at tumor mass. Therefore, drugs that derived from chimeric and humanized anti-human SM5-1 mAbs may be applied for tumor radiotherapy.

### Example 11

### Therapeutic Effects of ¹³¹I Labeled Anti-SM5-1 Antibody in Animal Model

Labeling of antibodies with Iodogen is mild, easy to do, with few injury and high efficiency of labeling.

1. Coating Reaction Tube

50 μl dichloride methane or chloroform containing 0.02% iodogen was added into the bottom of the reaction tube. The tube was dried with nitrogen or by air depression, and stored in dry condition at low temperature. The tube was washed several times with a few 0.05 mol/L, pH7.4 PBS before test to remove the reagent that failed to adhere.

2. Labeling Antibodies

The following substances were added into the reaction tube: Iodogen (0.02%), 50 μl; 0.05 mol/L, pH7.4 PBS, 50 μl; Na¹³¹, solution, 11 mCi/10 μl; antibody, 5-10 μg/μl. The solution was mixed up to allow reaction for 5-15 min at room temperature. The reaction was stopped by adding 200 μl of 0.05 mol/L, pH7.4 PBS.

3. Purifying Labeled Antibodies

The reaction mixture was loaded on a Sephadex G-50 gel column for gelfiltration purification. After testing, the radioactivity of the labeled antibody was 126 MBq/mg, which was enough for radioimmunotherapy.

4. Treating Tumor Cell Bearing Mouse with Radio-Labeled Antibodies

The method described above was used to label 5 mg purified chSM5-1 and ReSM5-1 antibodies. Thirty two tumor-bearing nude mice (bearing QYC cells) were randomly divided into 4 groups with 8 mice for each group (tumor mass about 0.7 cm in diameter). The labeled antibodies were injected into tail vein at a dosage of 5 GBq/kg for the therapeutic group. Eight weeks later, the tumor volume (if the animal died before the end of the experiment, measurement was made at death) and survival condition were recorded as shown in the following Table 6:

TABLE 6
Effects of ¹³¹I-labeled anti-SM5-1 antibodies
on tumor cells (QYC cells) bearing mice

| | Mean body | Mean tumor mass volume | Survival |
|---|---|---|---|
| Test group | weight (g) | (×10⁴ mm³) | rate |
| ¹³¹I-chSM5-1 | 18.6 ± 0.41 | 2.89 ± 0.14 | 7/8 |
| ¹³¹I-ReSM5-1 | 18.7 ± 0.65 | 3.23 ± 0.17 | 8/8 |
| ¹³¹I-CD3 | 19.1 ± 0.23 | 8.24 ± 0.83 | 3/8 |
| PBS | 21.7 ± 0.53 | 12.33 ± 0.55 | 0/8 |

The above results showed ¹³¹I labeled chimeric and humanized anti-human SM5-1 monoclonal antibodies were effective for tumor (hepatocellular carcinoma cells) bearing mice. These ¹³¹I, labeled antibodies reduced the tumor mass significantly and improved the survival rate of tumor-bearing mice.

### Example 12

### Comparison of Affinities Between Chimeric and Humanized Anti-SM5-1 Monoclonal Antibody

The Kds (Kon/Koff) of the two antibodies were determined with BIAcore from Pharmacia based on the approach provided by Karlsson et al. (Karlsson, R., Michaelsson, A., and Mattsson, L. 1991, Kinetic analysis of monoclonal antibody-antigen interactions with a new biosensor based analytical system. J. Immunol. Methods. 145, 229-240.) It was found that the Kds of the two antibodies were very similar with 9.31×10⁻⁹ M for humanized antibody and 3.78×10⁻⁹ M for the chimeric antibody.

The above results showed that the modulation for humanized anti-SM5-1 monoclonal was successful with almost no reduction of affinity and the affinity of the humanized antibody met the affinity demand for therapeutic monoclonal antibodies.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. An antibody that competitively inhibits the immunospecific binding of a human SM5-1 specific monoclonal antibody to a SM5-1 target antigen, wherein the variable region of heavy chain of said human SM5-1 specific monoclonal antibody comprises the amino acid sequence set forth in SEQ ID NO:9 and the variable region of light chain of said human SM5-1 specific monoclonal antibody comprises the amino acid sequence set forth in SEQ ID NO: 10.

2. The antibody of claim 1, which is selected from the group consisting of a polyclonal antibody, a monoclonal antibody, a Fab fragment, a Fab′ fragment, a F(ab′)₂ fragment, a Fv fragment, a diabody, a single-chain antibody and a multi-specific antibody formed from antibody fragments.

3. The antibody of claim 1, wherein the variable region of heavy chain of said antibody comprises the amino acid sequences 31-35, 50-66 and 99-108 set forth in SEQ ID NO:9 and the variable region of light chain of said antibody comprises the amino acid sequences 24-40, 56-62 and 95-102 set forth in SEQ ID NO:10.

4. The antibody of claim 3, wherein the variable region of heavy chain of the antibody comprises the amino acid sequence set forth in SEQ ID NO:9.

5. The antibody of claim 3, wherein the variable region of light chain of the antibody comprises the amino acid sequence set forth in SEQ ID NO: 10.

6. A human SM5-1 specific monoclonal antibody, wherein the variable region of heavy chain of the human SM5-1 specific monoclonal antibody comprises the amino acid sequence set forth in SEQ ID NO:9 and the variable region of light chain of the human SM5-1 specific monoclonal antibody comprises the amino acid sequence set forth in SEQ ID NO: 10.

7. An antibody that competitively inhibits the immunospecific binding of a SM5-1 specific monoclonal antibody to a SM5-1 target antigen, wherein the variable region of heavy chain of said SM5-1 specific monoclonal antibody comprises the amino acid sequence set forth in SEQ ID NO:1 and the variable region of light chain of said SM5-1 specific monoclonal antibody comprises the amino acid sequence set forth in SEQ ID NO:2.

8. The antibody of claim 7, which is selected from the group consisting of a polyclonal antibody, a monoclonal antibody, a Fab fragment, a Fab′ fragment, a F(ab′)₂ fragment, a Fv fragment, a diabody, a single-chain antibody and a multi-specific antibody formed from antibody fragments.

9. The antibody of claim 7, wherein the variable region of heavy chain of said antibody comprises the amino acid sequences 31-35, 50-66 and 99-108 set forth in SEQ ID NO:1 and the variable region of light chain of said antibody comprises the amino acid sequences 24-40, 56-62 and 95-102 set forth in SEQ ID NO:2.

10. The antibody of claim 9, which is a humanized antibody.

11. The antibody of claim 7, wherein the variable region of heavy chain of the antibody comprises the amino acid sequence set forth in SEQ ID NO:3 and the variable region of light chain of the antibody comprises the amino acid sequence set forth in SEQ ID NO:4.

12. A humanized SM5-1 specific monoclonal antibody, wherein the variable region of heavy chain of the humanized antibody comprises the amino acid sequence set forth in SEQ ID NO:1 and the variable region of light chain of the humanized antibody comprises the amino acid sequence set forth in SEQ ID NO:2.

13. An isolated nucleic acid comprising a nucleotide sequence encoding the heavy chain and/or the light chain, or a fragment thereof, of the antibody of claim 3.

14. An isolated nucleic acid comprising a nucleotide sequence encoding the heavy chain and/or the light chain, or a fragment thereof, of the human SM5-1 specific monoclonal antibody of claim 6.

15. The nucleic acid of claim 14, which comprises the nucleotide sequence set forth in SEQ ID NO:11 and/or SEQ ID NO:12.

16. An isolated nucleic acid comprising a nucleotide sequence complementary to the nucleotide sequence of claim 13.

17. A vector containing the nucleic acid of claim 13.

18. The vector of claim 17, which further comprises expression modulation sequence operatively linked to the nucleic acid encoding the heavy chain and/or the light chain, or a fragment thereof, of the antibody.

19. A recombinant cell containing the nucleic acid of claim 13.

20. The recombinant cell of claim 19, which is an eukaryote cell.

21. The recombinant cell of claim 19, which is a CHO cell.

22. An isolated nucleic acid comprising a nucleotide sequence encoding the heavy chain and/or the light chain, or a fragment thereof, of the antibody of claim 9.

23. An isolated nucleic acid comprising a nucleotide sequence encoding the heavy chain and/or the light chain, or a fragment thereof, of the humanized antibody of claim 12.

24. The nucleic acid of claim 23, which comprises the nucleotide sequence set forth in SEQ ID NO:5 and/or SEQ ID NO:6.

25. An isolated nucleic acid comprising a nucleotide sequence complementary to the nucleotide sequence of claim 22.

26. A vector containing the nucleic acid of claim 22.

27. The vector of claim 26, which further comprises expression modulation sequence operatively linked to the nucleic acid encoding the heavy chain and/or the light chain, or a fragment thereof, of the antibody.

28. A recombinant cell containing the nucleic acid of claim 22.

29. The recombinant cell of claim 28, which is an eukaryote cell.

30. The recombinant cell of claim 28, which is a CHO cell.

31. A method of producing a antibody, or a fragment thereof, comprising growing a recombinant cell containing the nucleic acid of claim 13 such that the encoded antibody, or a fragment thereof, is expressed by the cell, wherein the antibody is a human antibody; and recovering the expressed the antibody, or a fragment thereof.

32. The method of claim 31, which further comprises isolating and/or purifying the recovered antibody, or a fragment thereof.

33. A method of producing an antibody, or a fragment thereof, comprising growing a recombinant cell containing the nucleic acid of claim 22, such that the encoded antibody, or a fragment thereof, is expressed by the cell, wherein the antibody is a humanized antibody; and recovering the expressed antibody, or a fragment thereof.

34. The method of claim 33, which further comprises isolating and/or purifying the recovered antibody, or a fragment thereof.

35. A pharmaceutical composition comprising an effective amount of the antibody of claim 3 and a pharmaceutically acceptable carrier or excipient, wherein the antibody is a human antibody.

36. A pharmaceutical composition comprising an effective amount of a humanized SM5-1 specific monoclonal antibody and a pharmaceutically acceptable carrier or excipient, wherein the variable region of heavy chain of said humanized SM5-1 specific monoclonal antibody comprises the amino acid sequences 31-35, 50-66 and 99-108 set forth in SEQ ID NO: 1 and the variable region of light chain of said SM5-1 specific monoclonal antibody comprises the amino acid sequences 24-40, 56-62 and 95-102 set forth in SEQ ID NO:2.

37. A kit comprising an effective amount of an antibody of claim 3, and an instruction means for administering said antibody, wherein the antibody is a human antibody.

38. A kit comprising an effective amount of a humanized SM5-1 specific monoclonal antibody and an instruction means for administering said antibody, wherein the variable region of heavy chain of said humanized SM5-1 specific monoclonal antibody comprises the amino acid sequences 31-35, 50-66 and 99-108 set forth in SEQ ID NO:1 and the variable region of light chain of said humanized SM5-1 specific monoclonal antibody comprises the amino acid sequences 24-40, 56-62 and 95-102 set forth in SEQ ID NO:2.

39. A method for treating neoplasm in a mammal, which method comprises administering to a mammal to which such treatment is needed or desirable, an effective amount of the antibody of claim 1.

40. The method of claim 39, wherein the mammal is a human.

41. The method of claim 39, wherein the neoplasm is melanoma, breast cancer or hepatocellular carcinoma.

42. The method of claim 39, wherein the antibody is a human SM5-1 specific monoclonal antibody.

43. The method of claim 39, wherein the antibody exerts its anti-neoplasm effect via antibody dependent cell mediated cytotoxicity (ADCC) or complement dependent cell mediated cytotoxicity (CDC).

44. A method for treating neoplasm in a mammal, which method comprises administering to a mammal to which such treatment is needed or desirable, an effective amount of the antibody of claim 7.

45. The method of claim 44, wherein the antibody is a humanized antibody, and wherein the variable region of heavy chain of said humanized antibody comprises the amino acid sequences 31-35, 50-66 and 99-108 set forth in SEQ ID NO:1 and the variable region of light chain of said humanized antibody comprises the amino acid sequences 24-40, 56-62 and 95-102 set forth in SEQ ID NO:2.

46. The method of claim 45, wherein the mammal is a human.

47. The method of claim 45, wherein the neoplasm is melanoma, breast cancer or hepatocellular carcinoma.

48. The method of claim 45, wherein the humanized antibody exerts its anti-neoplasm effect via antibody dependent cell mediated cytotoxicity (ADCC) or complement dependent cell mediated cytotoxicity (CDC).

49. A combination, which combination comprises:
a) an effective amount of the antibody of claim 1; and
b) an effective amount of an anti-neoplasm agent.

50. The combination of claim 49, wherein the anti-neoplasm agent is an agent that treats melanoma, breast cancer or hepatocellular carcinoma.

51. A method for treating neoplasm in a mammal, which method comprises administering to a mammal to which such treatment is needed or desirable, an effective amount of a combination of claim 49.

52. A combination, which combination comprises:
a) an effective amount of the antibody of claim 7; and
b) an effective amount of an anti-neoplasm agent.

53. The combination of claim 52, wherein the antibody is a humanized antibody, and wherein the variable region of heavy chain of said humanized antibody comprises the amino acid sequences 31-35, 50-66 and 99-108 set forth in SEQ ID NO: 1 and the variable region of light chain of said humanized antibody comprises the amino acid sequences 24-40, 56-62 and 95-102 set forth in SEQ ID NO:2.

54. The combination of claim 52, wherein the anti-neoplasm agent is an agent that treats melanoma, breast cancer or hepatocellular carcinoma.

55. A method for treating neoplasm in a mammal, which method comprises administering to a mammal to which such treatment is needed or desirable, an effective amount of the combination of claim 52.

56. A method for inducing caspase-10 mediated apoptosis in a cell, which method comprises administering to a cell to which such induction is needed or desirable, an effective amount of the antibody of claim 1.

57. The method of claim 56, wherein the cell is a mammalian cell.

58. The method of claim 56, wherein the cell is contained in a mammal.

59. A method for inducing caspase-10 mediated apoptosis in a cell, which method comprises administering to a cell to which such induction is needed or desirable, an effective amount of the antibody of claim 7.

60. The method of claim 59, wherein the antibody is a humanized antibody, and wherein the variable region of heavy chain of said humanized antibody comprises the amino acid sequences 31-35, 50-66 and 99-108 set forth in SEQ ID NO:1 and the variable region of light chain of said humanized antibody comprises the amino acid sequences 24-40, 56-62 and 95-102 set forth in SEQ ID NO:2.

61. The method of claim 59, wherein the cell is a mammalian cell.

62. The method of claim 59, wherein the cell is contained in a mammal.

63. A conjugate, which conjugate comprises the antibody of claim 1 conjugated to a toxin and/or a radioactive isotope.

64. The conjugate of claim 63, wherein the antibody is a human antibody, and wherein the variable region of heavy chain of said antibody comprises the amino acid sequences 31-35, 50-66 and 99-108 set forth in SEQ ID NO:9 and the variable region of light chain of said antibody comprises the amino acid sequences 24-40, 56-62 and 95-102 set forth in SEQ ID NO: 10.

65. A conjugate, which conjugate comprises the antibody of claim 7 conjugated to a toxin and/or a radioactive isotope.

66. The conjugate of claim 65, wherein the antibody is a humanized antibody, and wherein the variable region of heavy chain of said humanized antibody comprises the amino acid sequences 31-35, 50-66 and 99-108 set forth in SEQ ID NO:1 and the variable region of light chain of said humanized antibody comprises the amino acid sequences 24-40, 56-62 and 95-102 set forth in SEQ ID NO:2.

67. A method for assaying for human SM5-1 target antigen in a sample, which method comprises:
a) obtaining a sample from a subject to be tested;
b) contacting said sample with an antibody of claim 1 under suitable conditions to allow binding between said human SM5-1 target antigen, if present in said sample, to said antibody; and
c) assessing binding between said human SM5-1 target antigen, if present in said sample, to said antibody to determine presence, absence and/or amount of said human SM5-1 target antigen in said sample.

68. The method of claim 67, which is used in the prognosis or diagnosis of a neoplasm.

69. The method of claim 68, wherein the neoplasm is melanoma, breast cancer or hepatocellular carcinoma.

70. A method for assaying for human SM5-1 target antigen in a sample, which method comprises:
a) obtaining a sample from a subject to be tested;
b) contacting said sample with the antibody of claim 7 under suitable conditions to allow binding between said human SM5-1 target antigen, if present in said sample, to said antibody; and
c) assessing binding between said human SM5-1 target antigen, if present in said sample, to said antibody to determine presence, absence and/or amount of said human SM5-1 target antigen in said sample.

71. The method of claim 70, which is used in the prognosis or diagnosis of a neoplasm.

72. The method of claim 71, wherein the neoplasm is melanoma, breast cancer or hepatocellular carcinoma.

73. A kit for assaying for human SM5-1 target antigen in a sample, which method comprises:
a) the antibody of claim 1; and
b) means for assessing binding between said human SM5-1 target antigen, if present in said sample, to said antibody to determine presence, absence and/or amount of said human SM5-1 target antigen in said sample.

74. A kit for assaying for human SM5-1 target antigen in a sample, which method comprises:
a) the antibody of claim 7; and
b) means for assessing binding between said human SM5-1 target antigen, if present in said sample, to said antibody to determine presence, absence and/or amount of said human SM5-1 target antigen in said sample.
